SEQUENCE LISTING (12 sequences)

SEQ ID NO: 1 - RNA, 19 nt, Artificial Sequence
  Direct repeat portion for Cas12 gRNA
aauuucuacuguuguagau

SEQ ID NO: 2 - RNA, 19 nt, Artificial Sequence
  Direct repeat portion for Cas12 gRNA
aauuucuacuauuguagau

SEQ ID NO: 3 - RNA, 23 nt, Artificial Sequence
  Direct repeat portion for Cas12 gRNA
auuuuugugcccaucguuggcac

SEQ ID NO: 4 - RNA, 18 nt, Artificial Sequence
  Direct repeat portion for Cas13 gRNA
gauuuagacuccccaaaa

SEQ ID NO: 5 - RNA, 16 nt, Artificial Sequence
  Direct repeat portion for Cas13 gRNA
gauuuagaacccaaaa

SEQ ID NO: 6 - RNA, 37 nt, Artificial Sequence
  Direct repeat portion for Cas14 gRNA
guugcagaacccgaauagacgaaugaaggaaugcaac

SEQ ID NO: 7 - RNA, 45 nt, Artificial Sequence
  gRNA
uaauuucuacuaaguguagaugagaagucauuuaauaaggccacu

SEQ ID NO: 8 - DNA, 24 bp, Artificial Sequence
  DNA target
gagaagtcatttaataaggccact

SEQ ID NO: 9 - DNA, 24 bp, Artificial Sequence
  DNA target
ctcttcagtaaattattccggtga

SEQ ID NO: 10 - DNA, 56 bp, Artificial Sequence
  Chimeric high Kd guide RNA with variable DNA sequence
  misc_feature (1)..(11): n is any suitable nucleotide (e.g., a, t, g, or c)
nnnnnnnnnnnuaauuucuacuaaguguagaugagaagucauuuaauaaggccacu

SEQ ID NO: 11 - DNA, 53 bp, Artificial Sequence
  Chimeric high Kd guide RNA with variable DNA sequence
  misc_feature (1)..(8): n is any suitable nucleotide (e.g., a, t, g, or c)
nnnnnnnnuaauuucuacuaaguguagaugagaagucauuuaauaaggccacu

SEQ ID NO: 12 - DNA, 55 bp, Artificial Sequence
  Circular chimeric guide RNA with variable DNA sequence
  misc_feature (1)..(10): n is any suitable nucleotide (e.g., a, t, g, or c)
nnnnnnnnnnuaauuucuacuaaguguagaugagaagucauuuaauaaggccacu

(19) United States
(12) Patent Application Publication
(10) Pub. No.: US 2024/0279719 A1
(43) Pub. Date: Aug. 22, 2024

(54) CRISPR CASCADE

(71) Applicant: The Board of Trustees of the University of Illinois, Urbana, IL (US)

(72) Inventor: Anurup Ganguli, Urbana, IL (US)

(73) Assignee: The Board of Trustees of the University of Illinois, Urbana, IL (US)

(21) Appl. No.: 18/570,745

(22) PCT Filed: Jun. 17, 2022
(86) PCT No.: PCT/US2022/033985

§ 371 Date: Dec. 15, 2023

Related U.S. Application Data
(60) Provisional application No. 63/279,357, filed on Nov. 15, 2021.
(60) Provisional application No. 63/211,642, filed on Jun. 17, 2021.

(51) Int. Cl. C12Q 1/6823; C12Q 1/6806; C12Q 1/689

(52) U.S. Cl. CPC C12Q 1/6823; C12Q 1/689; C12Q 1/6806

(57) ABSTRACT

Provided herein are compositions and methods that rapidly detect a target nucleic acid molecule using a CRISPR or a CRISPR Cascade mechanism where binding of a target nucleic acid molecule initiates an amplification cascade by converting high K_{D} guide RNA molecules to low K_{D} guide RNA molecules.

### PRIORITY

This application claims the benefit of U.S. Provisional application No. 63/211,642, filed on Jun. 17, 2021, and U.S. Provisional application No. 63/279,357, filed on Nov. 15, 2021, which are both incorporated herein in their entireties.

### STATEMENT REGARDING FEDERALLY SPONSORED RESEARCH OR DEVELOPMENT

This invention was made with Government support under RAPID Grant No. 2028431 awarded by the National Science Foundation. The Government has certain rights in this invention.

### INCORPORATION BY REFERENCE OF SEQUENCE LISTING

The instant application contains a Sequence Listing which has been submitted in ASCII format via EFS-Web and is hereby incorporated by reference in its entirety. Said ASCII copy, created on Jun. 7, 2022, is named “428262-000086_Sequence_Listing_ST25.txt” and is about 2.94 bytes in size.

### BACKGROUND

Techniques for detecting target nucleic acid sequences have far reaching research and clinical applications. Accurate and rapid nucleic-acid detection methods can facilitate early diagnosis and virus pandemic prevention. Currently, the demand is urgently increasing against the backdrop of the novel coronavirus SARS-COV-2, which has caused over 175 million cases and 3.81 million deaths worldwide (data as of 15 Jun. 2021). Detection methods can rely on pre-amplification of target nucleic acid molecules to enhance the detection sensitivity. However, pre-amplification can increase time to detection and change a sample to an extent that results generated from it are misleading or inaccurate. Methods are needed for rapid and direct detection of target nucleic acid sequences. Rapid and direct detection are essential for timely treatment, diagnosis, and research.

### SUMMARY

Provided herein are methods of detecting a target nucleic acid molecule in a sample. The methods comprise contacting the sample with:
(a) Cas RNA guided endonuclease molecules having RNA guided DNA or RNA endonuclease activity and indiscriminate ss RNA or ssDNA endonuclease activity;
(b) guide RNA (gRNA) specific for the target nucleic acid molecule;
(c) a signal amplification target nucleic acid molecule;
(d) a high K_{D} guide RNA (gRNA) specific for the signal amplification target nucleic acid molecule; and
(e) detecting a signal indicating presence of the target nucleic acid molecule. The signal amplification target nucleic acid molecule can comprise a nuclease resistant signal amplification target nucleic acid molecule. The high K_{D} guide RNA (gRNA) specific for the signal amplification target nucleic acid molecule can comprise a portion that is protected from nuclease activity. The high K_{D} gRNA can be configured to be cleaved by a first ribonucleoprotein complex comprising the Cas RNA guided endonuclease and the gRNA specific for the target nucleic acid molecule when the first ribonucleoprotein complex is activated by the target nucleic acid molecule. The cleavage of the high K_{D} gRNA by the first ribonucleoprotein complex can convert the high K_{D} gRNA into a low K_{D} gRNA. The low K_{D} gRNA can form a second ribonucleoprotein complex with the Cas RNA guided endonuclease, wherein the second ribonucleoprotein complex can be activated by the signal amplification target nucleic acid molecule. The high K_{D} gRNA can comprise a detectable signal moiety. The signal amplification target nucleic acid molecule can comprise one or more detectable signal moieties. The sample can further be contacted with one or more single stranded nucleic acid reporter molecules. The high K_{D} gRNA can comprise about 2 or more deoxyribonucleotides and about 45 ribonucleotides, wherein about 25 of the ribonucleotides are for Cas internalization and about 20 of the ribonucleotides are homologous to the target nucleic acid molecule. The about 2 or more deoxyribonucleotides can have one or more secondary structures. The high K_{D} gRNA can comprise about 2 or more deoxyribonucleotides and about 45 ribonucleotides, wherein about 25 of the ribonucleotides can be for Cas internalization and about 20 of the ribonucleotides can be homologous to the target nucleic acid molecule; wherein a 5′ end and a 3′ end of the high K_{D} gRNA can be covalently linked to form a circular molecule; the about 2 or more deoxyribonucleotides can be trans-cleavable and the about 45 ribonucleotides are not trans-cleavable. The methods can further comprise amplifying the target nucleic acid molecule prior to or along with the other steps of the method. The sample can comprise a minimally processed biological sample. The target nucleic acid molecule can be cDNA. The methods can use any of the high K_{D} gRNAs described herein. The Cas RNA guided endonuclease molecules and the guide RNA (gRNA) can be pre-assembled.

In other aspects compositions are provided. The compositions can comprise:
(a) Cas RNA guided endonuclease;
(b) gRNA specific for a target nucleic acid molecule;
(c) a signal amplification target nucleic acid molecule; and
(d) a high K_{D} gRNA specific for the signal amplification target nucleic acid molecule. The high K_{D} gRNA of the compositions can comprise any high K_{D} gRNAs described herein. Another embodiment provides a high K_{D} gRNA molecule comprising (i) a gRNA direct repeat portion of 15 to 40 ribonucleotides, (ii) a spacer portion of 17 to 26 ribonucleotides having homology to a target nucleic acid molecule, (iii) a portion that prevents formation of an active ribonucleoprotein (RNP) complex of 2 to 80 single stranded deoxyribonucleotides. The high K_{D} gRNA molecule can further comprise one or more detectable labels. The gRNA direct repeat portion and the spacer portion can comprise one or more modified internucleoside linkages configured to provide nuclease resistance to those portions. The one or more modified internucleoside linkages are phosphorothioate internucleoside linkages.

Yet another embodiment provides a high K_{D} gRNA molecule comprising (i) a gRNA direct repeat portion of 15 to 40 ribonucleotides, (ii) a spacer portion of 17 to 26 ribonucleotides with homology to a target nucleic acid molecule, (iii) a portion that prevents formation of an active ribonucleoprotein (RNP) complex of 2 to 80 single stranded ribonucleotides, wherein the gRNA direct repeat portion and/or the spacer portion comprise one or more modified internucleoside linkages configured to provide nuclease resistance to those portions. The one or more modified internucleoside linkages can be phosphorothioate internucleoside linkages. The high K_{D} gRNA molecule can further comprise one or more detectable labels.

Even another embodiment provides a circular high K_{D} gRNA molecule comprising (i) a gRNA direct repeat portion of 15 to 40 ribonucleotides, (ii) a spacer portion of 17 to 26 ribonucleotides having homology to a target nucleic acid molecule, and (iii) a portion that prevents formation of an active ribonucleoprotein (RNP) complex of 2 to 80 single stranded deoxyribonucleotides. The portion that prevents formation of an active ribonucleoprotein (RNP) complex can be cleavable by a Cas enzyme having indiscriminate single stranded deoxyribonucleotide cleavage activity. The direct repeat portion and the spacer portion can comprise one or more modified internucleoside linkages configured to provide nuclease resistance to those portions. The one or more modified internucleoside linkages can be phosphorothioate internucleoside linkages. The high K_{D} gRNA circular molecule can further comprising one or more detectable labels.

Another embodiment provides a high K_{D} gRNA circular molecule comprising a gRNA direct repeat portion of 15 to 40 ribonucleotides, (ii) a spacer portion of 17 to 26 ribonucleotides having homology to a target nucleic acid molecule, and (iii) a portion that prevents formation of an active ribonucleoprotein (RNP) complex of 2 to 80 single stranded ribonucleotides, wherein the gRNA direct repeat portion and/or the spacer portion can comprise one or more modified internucleoside linkages configured to provide nuclease resistance to those portions. The portion that prevents formation of an active ribonucleoprotein (RNP) complex can be cleavable by a Cas enzyme having indiscriminate single stranded ribonucleotide cleavage activity. The one or more modified internucleoside linkages can be phosphorothioate internucleoside linkages. The high K_{D} gRNA circular molecule can further comprise one or more detectable labels.

Yet another embodiment provides a high K_{D} gRNA molecule comprising (i) a gRNA direct repeat portion of 15 to 40 ribonucleotides, (iii) a spacer portion of 17 to 26 ribonucleotides with homology to a target nucleic acid molecule, (iii) a single stranded deoxyribonucleotide molecule hybridized to the gRNA direct repeat portion and the spacer region, wherein the single stranded deoxyribonucleotide molecule has 3 or more single stranded bulges of 2 or more deoxyribonucleotides, wherein the single stranded bulges can be cleaved by indiscriminate Cas enzyme mediated cleavage, and (iv) one or more detectable labels. The gRNA direct repeat portion and the spacer portion comprise one or more modified internucleoside linkages configured to provide nuclease resistance to those portions. The single stranded deoxyribonucleotide molecule can have 3 or more single stranded bulges of 2 or more deoxyribonucleotides. The three or more single stranded bulges can be cleavable by a Cas enzyme having indiscriminate single stranded cleavage activity.

Even another embodiment provides a high K_{D} gRNA molecule comprising (i) a gRNA direct repeat portion of 15 to 40 ribonucleotides, (iii) a spacer portion of 17 to 26 ribonucleotides with homology to a target nucleic acid molecule, (iii) a single stranded ribonucleotide molecule hybridized to the gRNA direct repeat portion and the spacer region, wherein the single stranded ribonucleotide molecule has 3 or more single stranded bulges of 2 or more ribonucleotides, wherein the single stranded bulges can be cleaved by indiscriminate Cas enzyme mediated cleavage, and (iv) one or more detectable labels. The gRNA direct repeat portion and the spacer portion can comprise one or more modified internucleoside linkages configured to provide nuclease resistance to those portions. The single stranded ribonucleotide molecule can have 3 or more single stranded bulges of 2 or more ribonucleotides. The three or more single stranded bulges can be cleavable by a Cas enzyme having indiscriminate single stranded cleavage activity.

Another embodiment provides a method of detecting a target nucleic acid molecule in a sample. The method comprises contacting the sample with (i) a preassembled complex comprising at least one guide RNA (gRNA) that is specific for the target nucleic acid molecule and a Cas protein having RNA guided DNA or RNA endonuclease activity and indiscriminate ssDNA or ssRNA endonuclease activity and (ii) a detectably labeled high K_{D} guide RNA (gRNA) that can be cleaved by the Cas protein after the preassembled complex is activated by the target nucleic acid molecule, wherein after cleavage, the high K_{D} guide RNA (gRNA) is (i) converted to a low K_{D} guide RNA (gRNA); and (ii) generates a detectable signal, whereby the target nucleic acid molecule is detected. The high K_{D} guide RNA can comprise any high K_{D} guide RNA as described herein. The low K_{D} guide RNA (gRNA) can bind to a ribonucleoprotein complex.

Therefore, provided herein are compositions and methods for technology that rapidly detects a target nucleic acid molecule using a CRISPR mechanism. In some embodiments the binding of the target nucleic acid molecule initiates an amplification cascade by converting high K_{D} guide RNA molecules to low K_{D} guide RNA molecules.

### BRIEF DESCRIPTION OF THE DRAWINGS

Various objectives, features, and advantages of the disclosed subject matter can be more fully appreciated with reference to the following detailed description of the disclosed subject matter when considered in connection with the following drawings, in which like reference numerals identify like elements.

FIG. 1 shows a flowchart for detecting a target nucleic acid molecule in one method using a CRISPR Cascade mechanism according to some embodiments where binding of the target nucleic acid molecule initiates an amplification cascade by converting high K_{D} guide RNA molecules low K_{D} guide RNA molecules. Step 1 comprises contacting an RNP1 complex with high K_{D} gRNA, signal amplification target nucleic molecule (target 2), and a target nucleic acid molecule (target 1). Step 2 demonstrates activation of the RNP1 complex upon binding to the target nucleic acid molecule. Upon activation, RNP1 has indiscriminate ssDNA endonuclease activity, which can cleave high K_{D} gRNA to form low K_{D} gRNA. Step 3 comprises assembly and activation of RNP2 complex. The assembled RNP2 complex is activated upon binding to signal amplification target nucleic acid molecule (target 2). Activation of the RNP2 complex begins indiscriminate ssDNA endonuclease activity, and a cascade of cleavage activity.

FIG. 2 shows step 4, a signal generation step.

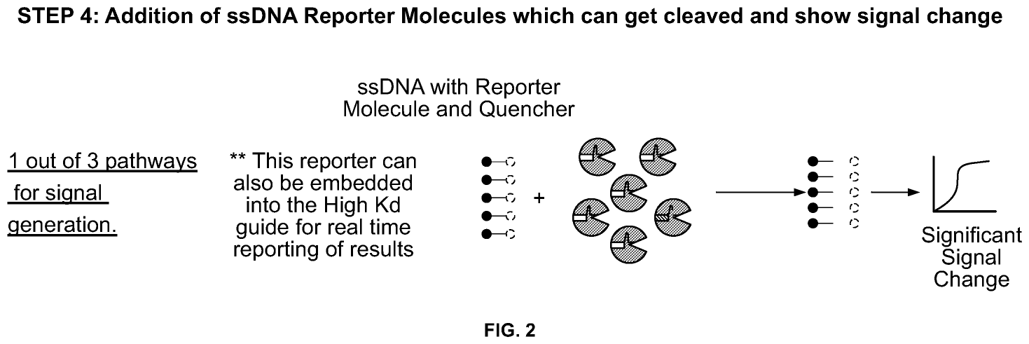

FIG. 3 shows three exemplary signal generation pathways: (1) a high K_{D} molecule converted to low K_{D} molecule that generates signal; (2) signal generation from signal amplification target nucleic acid molecule (target 2) cis-cleavage by activated RNP2; and (3) signal generation from ssDNA reporter trans-cleavage by activated RNP2.

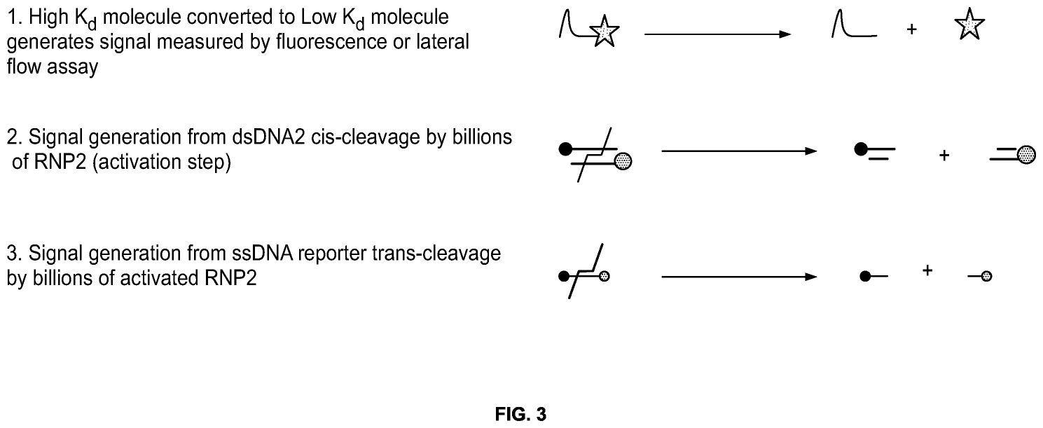

FIG. 4 shows exemplary guide RNA designs; UAAUUUCUACUAAGUGUAGA UGAGAAGUCAUUUAAUAAGGCCACU is SEQ ID NO:7; GAGAAGTCATTTAATAA GGCCACT is SEQ ID NO:8; CTCTTCAGTAAATTATTCCGGTGA is SEQ ID NO:9; NNNNNNNNNNNUAAUUUCUACUAAGUGUAGAUGAGAAGUCAUUUAAUAAGGCCACU is ID NO:10; and NNNNNNNNUAAUUUCUACUAAGUGUAGAUGAGAA SEQ GUCAUUUAAUAAGGCCACU is SEQ ID NO: 11.

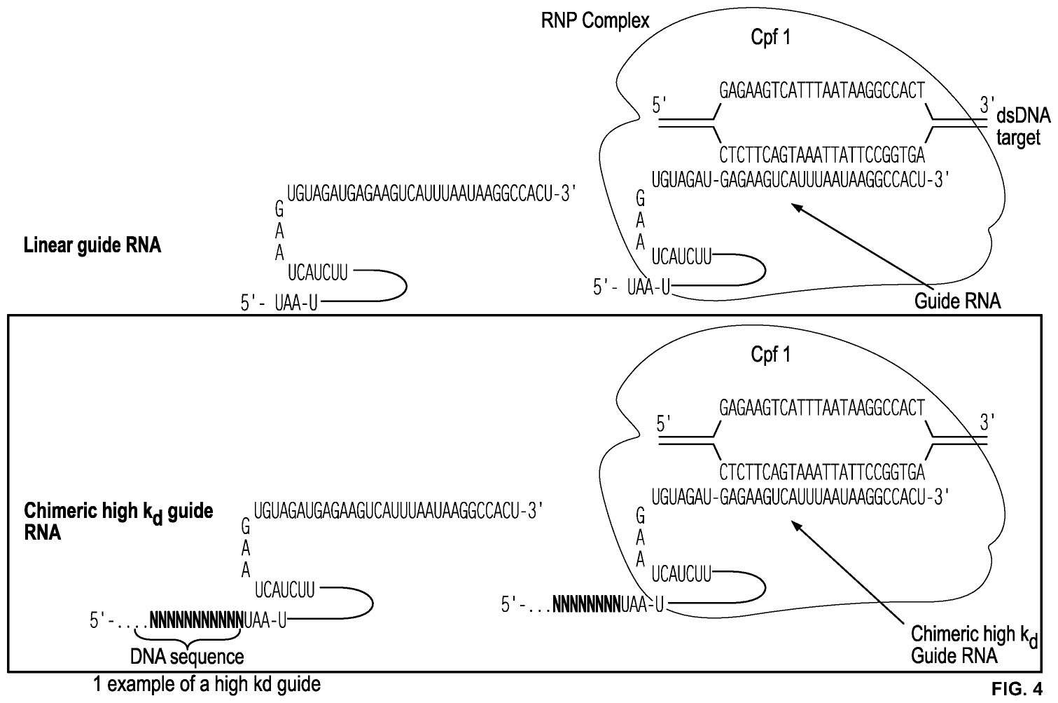

FIG. 5 shows high K_{D} guide RNA (linear and circular). Black lines show one phosphate linkage shown only for visualization of circular molecules; GAGAAGTCATTTAATAAGGCCACT is SEQ ID NO:8; CTCTTCAGTAAATTATTCCGGTGA is SEQ ID NO:9; and NNNNNNNNNNUAAUUUCUACUAAGUGUAGAUGAGAAGU CAUUUAAUAAGGCCACU is SEQ ID NO: 12.

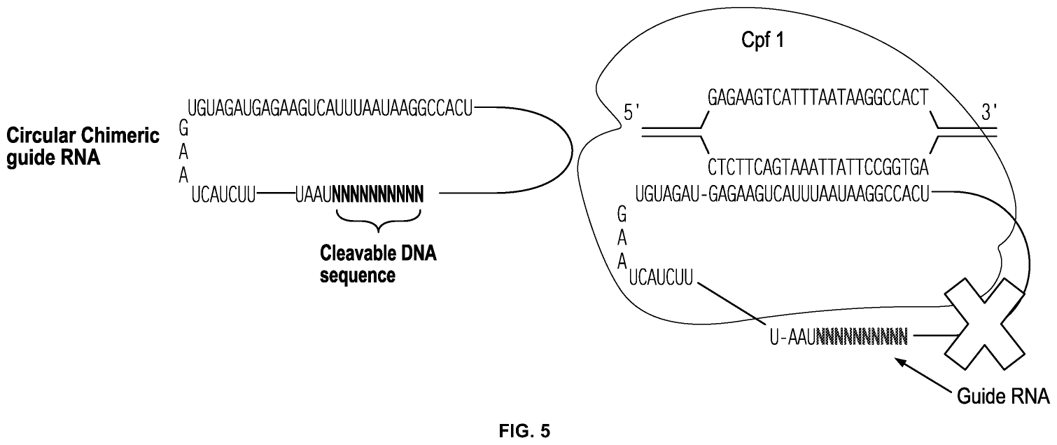

FIG. 6 shows a testing protocol.

FIGS. 7A-B show the successful detection of target nucleic acid molecules as compared to a negative control containing no copies of target nucleic acid molecules.

FIGS. 8A-B show the successful detection of target nucleic acid molecules as compared to a negative control containing no copies of target nucleic acid molecules.

FIGS. 9A-D show the successful detection of target nucleic acid molecules as compared to a negative control containing no copies of target nucleic acid molecules.

FIGS. 10A-B show the successful detection of target nucleic acid molecules as compared to a negative control containing no copies of target nucleic acid molecules.

FIGS. 11A-D show the successful detection of target nucleic acid molecules as compared to a negative control containing no copies of target nucleic acid molecules.

FIGS. 12A-C show the successful detection of target nucleic acid molecules as compared to a negative control containing no copies of target nucleic acid molecules.

The drawings are not necessarily to scale, or inclusive of all elements of a system, emphasis instead generally being placed upon illustrating the concepts, structures, and techniques sought to be protected herein.

### DETAILED DESCRIPTION

The details of one or more variations of the subject matter described herein are set forth in the accompanying drawings and the description below. Other features and advantages of the subject matter described herein will be apparent from the description and drawings, and from the claims. The disclosed subject matter is not, however, limited to any particular embodiment disclosed.

### Overview

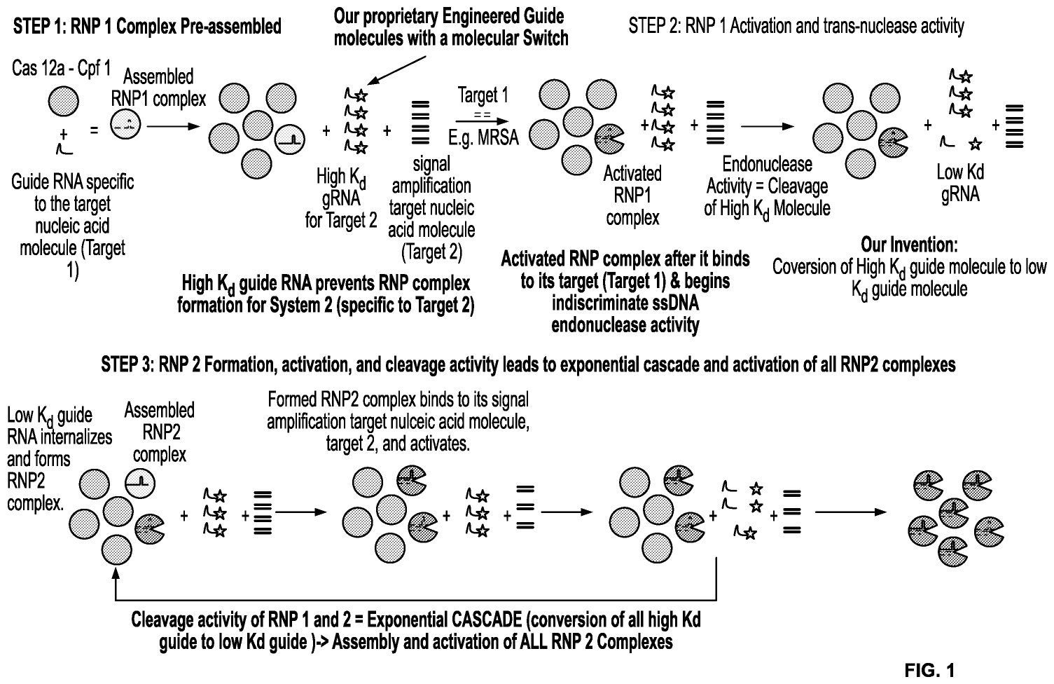

Compositions and methods for detecting a target nucleic acid molecule using a CRISPR s mechanisms and high K_{D} guide RNA (gRNA) are provided herein. In a first method a target nucleic acid molecule (target 1) in a sample can be detected by contacting the sample with (i) a preassembled complex comprising at least one guide RNA (gRNA) that is specific for the target nucleic acid molecule and a Cas protein having RNA guided DNA or RNA endonuclease activity and indiscriminate ssDNA or ssRNA endonuclease activity and (ii) a detectably labeled high K_{D} guide RNA (gRNA) that can be cleaved by the Cas protein after the preassembled complex is activated by the target nucleic acid molecule, wherein after cleavage, the high K_{D} guide RNA (gRNA) is (i) converted to a low K_{D} guide RNA (gRNA); and (ii) generates a detectable signal, whereby the target nucleic acid molecule is detected. See Step 1 and Step 2 of FIG. 1 (however, the signal amplification target nucleic acid molecule is not required or used in these methods). In these methods the target nucleic acid can optionally be amplified prior to detection.

For example, a sample can be contacted with a preassembled complex comprising at least one guide RNA (gRNA) that is specific for the target nucleic acid molecule and a Cas protein having RNA guided DNA or RNA endonuclease activity and indiscriminate ssDNA or ssRNA endonuclease activity. Alternatively, at least one guide RNA (gRNA) that is specific for the target nucleic acid molecule and a Cas protein having RNA guided DNA or RNA endonuclease activity and indiscriminate ssDNA or ssRNA endonuclease activity can be provided separately. A detectably labeled high K_{D} guide RNA (gRNA) that can be cleaved by the Cas protein after the preassembled complex is activated by the target nucleic acid molecule is also added to the sample. After cleavage, the high K_{D} guide RNA (gRNA) can be (i) converted to a low K_{D} guide RNA (gRNA); and/or (ii) generates a detectable signal, whereby the target nucleic acid molecule is detected.

The Cas protein can be any suitable Cas protein having indiscriminate single stranded cleavage activity. The resulting low K_{D} guide RNA (gRNA) can also bind to another second ribonucleoprotein complex in the assay as described below.

In additional methods, the signals can be amplified in a cascading mechanism so that a greater signal is produced. See Step 1, Step 2, and Step 3 of FIG. 1. In this method, a first ribonucleoprotein (RNP) complex, RNP1, is formed from a Cas enzyme, such as Cas12a, and gRNA specific to a target nucleic acid molecule (target 1). The Cas enzyme and gRNA can be added as a preassembled RNP1 complex or as separate components to the assay. The binding of a target nucleic acid molecule activates the RNP1 complex to have indiscriminate ssDNA endonuclease activity. The indiscriminate ssDNA or ssRNA endonuclease activity cleaves high K_{D} gRNA to form low K_{D} gRNA. In its uncleaved form, the high K_{D} gRNA can prevent assembly of an RNP2 complex. However, the low K_{D} gRNA formed following cleavage by the activated RNP1 complex forms a second ribonucleoprotein complex, RNP2. The RNP2 complex comprises a Cas enzyme and a low K_{D} gRNA specific for a signal amplification target nucleic acid molecule for CRISPR Cascade, target 2. The RNP2 complex binds to its signal amplification target nucleic acid molecule (target 2) thereby activating the RNP2 complex. The activated RNP2 complex then has indiscriminate ssDNA or ssRNA endonuclease activity, which can form more low K_{D} gRNA molecules, which assemble more RNP2 complexes. The assembled RNP2 complexes activate by binding to the signal amplification target nucleic acid molecule (target 2) and, along with activated RNP1, cleave a high K_{D} gRNA to form low K_{D} gRNA. Reporter molecules can be introduced to elements of the system to indicate cleavage, thereby detecting target nucleic acid molecules. In these methods it is not necessary to amplify the target nucleic acids prior to first step of the assay.

### Polynucleotides

Polynucleotides or nucleic acid molecules are a series of nucleotide bases: deoxyribonucleic acid (DNA) or ribonucleic acid (RNA). Nucleic acid molecules include but are not limited to genomic DNA, cDNA, mRNA, IRNA, miRNA, tRNA, ncRNA, rRNA, DNA-RNA hybrid sequences and recombinantly produced and chemically synthesized molecules such as aptamers, plasmids, antisense DNA strands, shRNA, ribozymes, nucleic acids conjugated, oligonucleotides or combinations thereof. Unless otherwise indicated, the term polynucleotide, nucleic acid molecule, or gene includes reference to the specified sequence as well as the complementary sequence thereof. Polynucleotides can be present as a single-stranded or double-stranded and linear or covalently circularly closed molecule. As used herein, a polynucleotide can include both naturally occurring and non-naturally occurring nucleotides.

Polynucleotides can be obtained from nucleic acid molecules present in, for example, a mammalian cell. Polynucleotides can also be synthesized in the laboratory, for example, using an automatic synthesizer. Polynucleotides can be isolated. An isolated polynucleotide can be a naturally occurring polynucleotide that is not immediately contiguous with one or both of the 5′ and 3′ flanking genomic sequences that it is naturally associated with. An isolated polynucleotide can be, for example, a recombinant DNA molecule of any length, provided that the nucleic acid molecules naturally found immediately flanking the recombinant DNA molecule in a naturally occurring genome is removed or absent. Isolated polynucleotides also include non-naturally occurring nucleic acid molecules. “Isolated polynucleotides” can be (i) amplified in vitro, for example via polymerase chain reaction (PCR), (ii) produced recombinantly by cloning, (iii) purified, for example, by cleavage and separation by gel electrophoresis, (iv) synthesized, for example, by chemical synthesis, or (vi) extracted from a sample.

Polynucleotides can encode full-length polypeptides, polypeptide fragments, and variant or fusion polypeptides. Polynucleotides can comprise coding sequences for naturally occurring polypeptides or can encode altered sequences that do not occur in nature. Polynucleotides can be purified free of other components, such as proteins, lipids and other polynucleotides. For example, the polynucleotide can be 50%, 75%, 90%, 95%, 96%, 97%, 98%, 99%, or 100% purified. A polynucleotide existing among hundreds to millions of other polynucleotide molecules within, for example, cDNA or genomic libraries, or gel slices containing a genomic DNA restriction digest are not to be considered a purified polynucleotide.

### RNP Complexes

CRISPR systems perform interference of a target nucleic acid molecule by coupling gRNAs and Cas enzymes, thereby forming CRISPR ribonucleoproteins (RNP) complexes. Generally, the gRNA of the RNP guides the RNP to a nucleic acid molecule, (e.g., by recognizing the nucleic acid molecule through hybridization). Hybridized target nucleic acid molecule-gRNA units are subjected to cleavage by Cas proteins. Target nucleic acid molecule interference typically requires a protospacer adjacent motif (PAM) in a target nucleic acid molecule.

CRISPR genome-editing tools described herein comprise a gRNA specific for a nucleic acid molecule and a Cas endonuclease. The gRNA and Cas endonuclease can be used as a preassembled RNP in an assay or the gRNA and Cas endonuclease can be added independently to an assay. The nucleic acid molecule can be a target nucleic acid molecule (target 1, i.e., the nucleic acid molecule to be detected in a sample such as a biological sample) and/or a signal amplification target nucleic acid molecule (target 2) for CRISPR Cascade. A gRNA specific for a nucleic acid molecule and a Cas endonuclease are the two components that form the RNP complex that recognizes target sequences accompanying a protospacer-adjacent motif (PAM), subsequently inducing a double-stranded break (DSB) either inside or outside the protospacer region on the nucleic acid molecule [the target nucleic acid molecule (target 1) or the signal amplification target nucleic acid molecule (target 2)]. In some embodiments, an RNP complex can be RNP1, which is comprised of gRNA specific to the target nucleic acid molecule (target 1) and a Cas enzyme. In some embodiments, an RNP complex can be RNP2, which is comprised of gRNA specific to the signal amplification target nucleic acid molecule (target 2) and a Cas enzyme. Cas enzymes are described in greater detail below.

In some embodiments, an RNP complex is pre-assembled. An RNP complex, such as RNP1 or RNP2, is pre-assembled when a gRNA specific for the target nucleic acid molecule (target 1) is integrated with the Cas enzyme. In some embodiments, a gRNA that is specific for a target nucleic acid molecule (target 1) and a Cas enzyme from a RNP1 complex. In some embodiments, a low K_{D} gRNA specific for a signal amplification target nucleic acid molecule (target 2) and a Cas enzyme form a RNP2 complex. A low K_{D} gRNA results when a high K_{D} gRNA is cleaved as discussed below.

In some embodiments, a method of detecting a target nucleic acid molecule (target 1) can comprise using a pre-assembled RNP1 complex. An RNP1 complex can be pre-assembled in that the gRNA specific for the target nucleic acid molecule (target 1) and Cas enzyme have already formed a RNP complex in the initial reaction composition. In some embodiments, the initial reaction composition contains a pre-assembled RNP1 complex, a high K_{D} gRNA and corresponding signal amplification target nucleic acid molecule (target 2) for potential formation of RNP2. These elements are discussed in greater detail below.

In some embodiments, an RNP complex is activated. An RNP complex, such as RNP1 or RNP2, is activated when the gRNA specific for the target of nucleic acid molecule (target 1) binds to or hybridizes with the target nucleic acid molecule (target 1) and begins endonuclease activity. An activated RNP complex can exhibit the characteristic cleavage activity of a suitable Cas enzyme such as Cas12a, as discussed below. In some embodiments, binding of the target nucleic acid molecule (target 1) activates an RNP1 complex. In some embodiments, binding of a signal amplification target nucleic acid molecule (target 2) for CRISPR Cascade activates an RNP2 complex.

### Target Sequence

In the context of formation of a CRISPR complex, a target sequence (e.g., a target nucleic acid molecule or a signal amplification target DNA molecule) is a sequence to which a guide sequence is designed to have complementarity, where hybridization between a target sequence and a guide sequence promotes the formation of a CRISPR complex. A guide sequence can be gRNA, as discussed below. A CRISPR complex can be, for example, an RNP complex as descried herein, such as RNP1 or RNP2. Full complementarity is not necessarily required, provided there is sufficient complementarity to cause hybridization and promote formation of a CRISPR complex. A target sequence can comprise any polynucleotide, such as DNA (ssDNA or dsDNA) or RNA (ssRNA or dsRNA) polynucleotides. In some embodiments, a target sequence is located in the nucleus or cytoplasm of a cell. In some embodiments, the target sequence can be within an organelle of a eukaryotic cell, for example, mitochondrion or chloroplast. In some embodiments, a target sequence can be a target nucleic acid molecule (target 1) or a signal amplification target nucleic acid molecule (target 2).

The degree of complementarity between a guide sequence and its corresponding target sequence (target 1 or target 2, discussed in detail below), when optimally aligned using a suitable alignment algorithm, is about 50%, 60%, 75%, 80%, 85%, 90%, 95%, 97.5%, 99%, or more. Optimal alignment can be determined with the use of any suitable algorithm for aligning sequences, non-limiting example of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies, ELAND (Illumina, San Diego, Calif.), SOAP (available at soap.genomics.org.cn), and Maq (available at m aq.sourceforge.net).

### Target Nucleic Acid Molecules

The target nucleic acid molecule (target 1) of a CRISPR complex can be any polynucleotide endogenous or exogenous to a host cell, such as a eukaryotic cell or a prokaryotic cell. In an embodiment, a target nucleic acid molecule (target 1) is present in a sample, such as a biological sample. In another embodiment, a target nucleic acid molecule (target 1) can be a viral nucleic acid molecule or a nucleic acid not associated with a cell. The target nucleic acid molecule (target 1) can be a polynucleotide, which can be a sequence coding a gene product (e.g., a protein) or a non-coding sequence (e.g., a regulatory polynucleotide). The target nucleic acid molecule (target 1) can be associated with a PAM (protospacer adjacent motif); that is, a short sequence recognized by the CRISPR complex. The precise sequence and length requirements for the PAM differ depending on the RNA-guided endonuclease used, but PAMs are typically 2-5 base pair sequences adjacent to the protospacer (that is, the target sequence). Those of ordinary skill in the art skilled can identify PAM sequences for use with a given RNA-guided DNA endonuclease enzyme.

Binding of target nucleic acid molecules (target 1) to complementary gRNA in an RNP1 complex precipitates a cascade mechanism. Target nucleic acid molecules (target 1) are nucleic acid segments of interest. Target nucleic acid molecules can include DNA molecules (e.g., a cDNA or genomic DNA), RNA molecules (e.g., an mRNA), tRNA, or rRNA. The target nucleic acid molecule (target 1) can be single-stranded or double-stranded. Such target nucleic acid molecule (target 1) can be coding or non-coding nucleic acid molecules. In some embodiments, the target nucleic acid molecule (target 1) of interest is DNA. In some embodiments, the target nucleic acid molecule (target 1) is RNA converted to cDNA prior to detection. In some embodiments 1, 2, 3, 4, 5, 6, 7, 8, 9, 10, or more target nucleic acid molecules (target 1) can be detected in a single assay.

In an embodiment a target nucleic acid molecule is from a Eukaryote, for example a gene or portion of a gene, a non-coding region, a promoter, an intron, or an untranslated region. In another embodiment a target nucleic acid molecule is from a virus, for example, hepatitis B virus (HBV), hepatitis C virus (HCV), hepatitis D virus hepatitis E virus (HEV), human immunodeficiency virus (HIV), West Nile virus (WNV), and Dengue virus (DENV), Coronavirus, Severe Acute Respiratory Syndrome Coronavirus 2 (SARS-COV-2), Middle East Respiratory Syndrome Coronavirus (MERS-COV), Severe Acute Respiratory Syndrome Coronavirus (SARS-CoV), Respiratory Syncytial Virus (RSV), Rhinovirus, Influenza A, Influenza B, Influenza C, Human metapneumovirus, LCMV (lymphocytic choriomeningitis virus), Coxsackie B virus (CBV), Parainfluenza virus type 1, Parainfluenza virus type 2, Parainfluenza virus type 3, Parainfluenza virus type 4, Adenovirus, Enterovirus, Varicella-zoster virus, Hantavirus, Epstein-Barr virus (EBV), Herpes Simplex Virus, Cytomegalovirus (CMV), Human metapneumovirus, Poliovirus, Rhinovirus, Norwalk virus, Yellow fever virus, Enterovirus, Zika virus, Rubella virus, Ross River virus, Sindbis virus, Chikungunya virus, Borna disease virus, Ebola virus, Marburg virus, Measles virus, Mumps virus, Nipah virus, Hendra virus, Newcastle disease virus, Human respiratory syncytial virus, Rabies virus, Lassa virus, Hantavirus, Crimean-Congo hemorrhagic fever virus, Human parainfluenza viruses 1-4.

A target nucleic acid molecule can be a bacterial nucleic acid molecule from, for example, methicillin-susceptible and methicillin-resistant staphylococci (including Staphylococcus aureus, Staphylococcus epidermidis, Staphylococcus haemolyticus, Staphylococcus hominis, Staphylococcus saprophyticus, and coagulase-negative staphylococci), glycopeptides-intermediate susceptible Staphylococcus aureus, penicillin-susceptible and penicillin-resistant streptococci (including Streptococcus pneumoniae, Streptococcus pyogenes, Streptococcus agalactiae, Streptococcus avium, Streptococcus bovis, Streptococcus lactis, Streptococcus sanguis and Streptococci Group C, Streptococci Group G and viridans streptococci), Enterococci (including vancomycin susceptible and vancomycin-resistant strains such as Enterococcus faecalis and Enterococcus faecium), Clostridium difficile, Listeria monocytogenes, Corynebacterium jeikeium, Chlamydia spp. (including C. pneumoniae) and Mycobacterium tuberculosis, Enterobacteriacae, including Escherichia spp. (including Escherichia coli), Klebsiella spp., Enterobacter spp., Citrobacter spp., Serratia spp., Proteus spp., Providencia spp., Salmonella spp., Shigella spp., Pseudomonas (including P. aeruginosa), Moraxella spp. (including M. catarrhalis), Haemophilus spp. and Neisseria spp., Pseudomonas fluorescens, Pseudomonas acidovorans, Pseudomonas alcaligenes, Pseudomonas putida, Stenotrophomonas maltophilia, Burkholderia cepacia, Aeromonas hydrophilia, Citrobacter freundii, Salmonella typhimurium, Salmonella typhi, Salmonella paratyphi, Salmonella enteritidis, Shigella dysenteriae, Shigella flexneri, Shigella sonnei, Enterobacter cloacae, Enterobacter aerogenes, Klebsiella pneumoniae, Klebsiella oxytoca, Serratia marcescens, Francisella tularensis, Morganella morganii, Proteus mirabilis, Proteus vulgaris, Providencia alcalfaciens, Providencia rettgeri, Providencia stuartii, Acinetobacter baumannii, Acinetobacter calcoaceticus, Acinetobacter haemolyticus, Yersinia Yersinia enterocolitica, pestis, Yersinia pseudotuberculosis, Yersinia intermedia, Bordetella pertussis, Bordetella parapertussis, Bordetella bronchiseptica, Haemophilus influenzae, Haemophilus parainfluenzae, Haemophilus haemolyticus, Haemophilus parahaemolyticus, Haemophilus ducreyi, Pasteurella multocida, Pasteurella haemolytica, Branhamella catarrhalis, Helicobacter pylori, Campylobacter fetus, Campylobacter jejuni, Campylobacter coli, Borrelia burgdorferi, Vibrio cholerae, Vibrio parahaemolyticus, Legionella pneumophila, Listeria monocytogenes, Neisseria gonorrhoeae, Neisseria meningitidis, Kingella, Moraxella, Gardnerella vaginalis, Bacteroides fragilis, Bacteroides distasonis, Bacteroides 3452A homology group, Bacteroides vulgatus, Bacteroides ovalus, Bacteroides thetaiotaomicron, Bacteroides unformis, Bacteroides eggerthii, Bacteroides splanchnicus, Clostridium difficile, Mycobacterium tuberculosis, Mycobacterium avium, Mycobacterium intracellulare, Mycobacterium leprae, Corynebacterium diphtheriae, Corynebacterium ulcerans, Streptococcus pneumoniae, Streptococcus agalactiae, Streptococcus pyogenes, Enterococcus faecalis, Enterococcus faecium, Staphylococcus aureus, Staphylococcus epidermidis, Staphylococcus saprophyticus, Staphylococcus intermedius, Staphylococcus hyicus subsp. hyicus, Staphylococcus haemolyticus, Staphylococcus hominis, and Staphylococcus saccharolyticus.

A target nucleic acid molecule can be a nucleic acid molecule can be from a parasite such as those from phylum Apicomplexa, Sarcomastigophora (including Trypanosoma, Plasmodia, Leishmania, Babesia or Theileria), Cryptosporidia, Sacrocystida, Amoebia, Coccidia and Trichomonadia.

### RNA Guided DNA or RNA Endonucleases

Methods of detecting a target nucleic acid molecule (target 1) in a sample can comprise contacting the sample with an RNA guided DNA or RNA endonuclease such as a Cas enzyme. An RNA guided RNA or DNA endonuclease is a CRISPR-associated protein that is an RNA-guided endonuclease suitable for RNP complex formation with sequence specific gRNA. In some embodiments, a Cas RNA guided endonuclease can be a Cas RNA guided DNA endonuclease, such as but not limited to Cas12a, Cas12b, Cas12c, Cas12d, Cas12e, Cas14, Cas12h, Cas12i, or Cas12j. In some embodiments, a Cas RNA guided endonuclease can be a Cas RNA guided RNA endonuclease, such as Cas13a or Cas12g. In some embodiments, a Cas RNA guided endonuclease can be any nucleic acid (DNA or RNA) targeting Cas endonuclease with collateral trans-cleavage activity.

The present disclosure provides compositions and methods that take advantage type V CRISPR/Cas proteins (e.g., Cas 12 proteins such as Cas12a (formerly Cpf1) and Cas12b (C2c1)), which can indiscriminately cleave non-targeted single stranded DNA (ssDNA) once activated by detection of a target nucleic acid molecule (target 1). Once a type V CRISPR/Cas effector protein (e.g., a Cas12 protein such as Cas12a, Cas12b, Cas12c, Cas12d, Cas12e) is activated by gRNA, which occurs when a sample includes a target nucleic acid molecule (target 1) to which the gRNA hybridizes (i.e., the sample includes the targeted nucleic acid molecule), the protein becomes a nuclease that indiscriminately cleaves ssDNAs or ssRNAs (i.e., non-target ssDNAs or ssRNAs to which a guide sequence for a target nucleic acid molecule does not hybridize). Furthermore, nuclease activity involves cleavage of the non-target strand of the target nucleic acid molecule. Thus, when a target nucleic acid molecule (target 1) is present in the sample, one result is cleavage of ssDNAs in the sample, which can be detected using e.g., a labeled single stranded detector DNA or RNA, or any other suitable detection method.

Type V CRISPR/Cas effector proteins are a subtype of Class 2 CRISPR/Cas effector proteins. For examples of type V CRISPR/Cas systems and their effector proteins (e.g., Cas12 family proteins such as Cas12a), see, e.g., Shmakov et al., Nat Rev Microbiol. 2017 March; 15(3): 169-182: “Diversity and evolution of class 2 CRISPR-Cas systems.” In some embodiments, methods of detecting a target nucleic acid molecule (target 1) can use other CRISPR/Cas effector proteins with functionality similar to that of Cas12a. Examples include, but are not limited to: Cas12 family (Cas12a, Cas12b, Cas12c), C2c4, C2c8, C2c5, C2c10, and C2c9; as well as CasX (Cas12e) and CasY (Cas12d). Also see, e.g., Koonin et al., Curr Opin Microbiol. 2017 June; 37:67-78: “Diversity, classification and evolution of CRISPR-Cas systems.”

As such in some cases, a subject type V CRISPR/Cas effector protein is a Cas12 protein (e.g., Cas12a, Cas12b, Cas12c). In some cases, a subject type V CRISPR/Cas effector protein is a Cas12 protein such as Cas12a, Cas12b, Cas12c, Cas12d, Cas12e, Cas12d, or Cas12e. In some cases, a subject type V CRISPR/Cas effector protein is a Cas12a protein. In some cases, a subject type V CRISPR/Cas effector protein is a Cas12b protein. In some cases, a subject type V CRISPR/Cas effector protein is a Cas12c protein. In some cases, a subject type V CRISPR/Cas effector protein is a Cas12d protein. In some cases, a subject type V CRISPR/Cas effector protein is a Cas12e protein. In some cases, a subject type V CRISPR/Cas effector protein is protein selected from: Cas12 (e.g., Cas12a, Cas12b, Cas12c, Cas12d, Cas12e), C2c4, C2c8, C2c5, C2c10, and C2c9. In some cases, a subject type V CRISPR/Cas effector protein is protein selected from: C2c4, C2c8, C2c5, C2c10, and C2c9. In some cases, a subject type V CRISPR/Cas effector protein is protein selected from: C2c4, C2c8, and C2c5. In some cases, a subject type V CRISPR/Cas effector protein is protein selected from: C2c10 and C2c9.

In some cases, the subject type V CRISPR/Cas effector protein is a naturally-occurring protein (e.g., naturally occurs in prokaryotic cells). In other cases, the Type V CRISPR/Cas effector protein is not a naturally-occurring polypeptide (e.g., the effector protein is a variant protein, a chimeric protein, includes a fusion partner, and the like). Examples of naturally occurring Type V CRISPR/Cas effector proteins include, but are not limited to Cas12a that can be the isolated from, for example, Francisella tularensis subsp. novicida (Gene ID: 60806594), Candidatus Methanoplasma termitum (Gene ID: 24818655), Candidatus Methanomethylophilus alvus (Gene ID: 15139718), or Eubacterium eligens ATCC 27750 (Gene ID: 41356122). In some embodiments, Cas12a endonucleases with altered PAM specificity can be used such as those described in WO2018195545A2. In some embodiments, the Cas12a protein can any one of those described in US20200299768A1.

Any Type V CRISPR/Cas effector protein can be suitable for the compositions and methods of the present disclosure as long as the Type V CRISPR/Cas effector protein forms a complex with a guide RNA and exhibits suitable cleavage activity. For example, suitable cleavage activity can be ssDNA or ssRNA cleavage of non-target ssDNAs or ssRNAs once the complex (RNP1 or RNP2) is activated by hybridization of an associated guide RNA to its target nucleic acid molecule (target 1). In some embodiments, a Cas13a endonuclease can be suitable for RNA-guided RNA cleavage by Cas13a.

In some embodiments, an RNA-guided endonuclease protein, e.g., Cas12a, is directed to a specific nucleic acid target by a gRNA, where it causes a double-strand break. In some embodiments, the target nucleic acid molecule (target 1) is dsDNA. In some embodiments, an RNA-guided endonuclease binds to its target sequence in the presence of a protospacer adjacent motif (PAM) on the non-targeted DNA strand. An RNA-guided DNA endonuclease cuts 3-4 nucleotides upstream of the PAM sequence. Recognition of the PAM sequence by an RNA-guided DNA endonuclease protein is thought to destabilize the adjacent DNA sequence, allowing interrogation of the sequence by the sgRNA, and allowing the sgRNA-DNA pairing when a matching sequence is present.

In some embodiments, the RNA-guided endonuclease directs cleavage of both strands of target nucleic acid molecule (target 1) within about 1, 2, 3, 4, 5, 6, 7, 8, 9, 10, 15, 20, 25, 50, 100, 200, 500, or more base pairs from the first or last nucleotide of a target sequence.

In some embodiments, Cas12a cleaves both the target and non-target strands of a targeted dsDNA by a single active site in the RuvC catalytic pocket. This activity is referred to as cis-cleavage of the target dsDNA. Type V CRISPR/Cas proteins, e.g., Cas12 proteins such as Cas12a, also can perform trans-cleavage or collateral cleavage of non-targeted single-stranded DNA (ssDNA) upon activation of an RNP complex, such as RNP1 or RNP2, by binding of a target nucleic acid molecule, such as the target nucleic acid molecule (target 1) in the case of RNP1 and the signal amplification target nucleic acid molecule (target 2) for CRISPR Cascade in the case of RNP2. Additionally, Cas12a can cleave a target nucleic acid molecule that is single stranded while also performing trans-cleavage or collateral cleavage of non-targeted ssDNA upon activation of the RNP complex. In some embodiments, Cas12a RNA guided endonuclease forms a first ribonucleoprotein complex, RNP1, with gRNA specific for a target nucleic acid molecule (target 1). In some embodiments, one or more Cas12a RNA guided endonucleases form a second ribonucleoprotein complex, RNP2, with high K_{D} gRNA specific for signal amplification target nucleic acid molecule (target 2). The specificity of high K_{D} gRNA is discussed below. In some embodiments, Cas12a RNA guided endonucleases form both RNP1 and RNP2 complexes.

In some embodiments, an RNA-guided RNA endonuclease protein, e.g., Cas13a, is directed to a specific nucleic acid target by a gRNA, where it causes a double-strand break. In some embodiments, the target nucleic acid molecule (target 1) is dsRNA.

In some embodiments, the RNA guided RNA endonuclease directs cleavage of both strands of target nucleic acid molecule (target 1) within about 1, 2, 3, 4, 5, 6, 7, 8, 9, 10, 15, 20, 25, 50, 100, 200, 500, or more base pairs from the first or last nucleotide of a target sequence.

In some embodiments, Cas13a cleaves targeted RNA. Cas13a (and other RNA guided RNA endonucleases) also can perform trans-cleavage or collateral cleavage of non-targeted single-stranded RNA (ssRNA) upon activation of an RNP complex, such as RNP1 or RNP2, by binding of a target nucleic acid molecule, such as the target nucleic acid molecule (target 1) in the case of RNP1 and the signal amplification target nucleic acid molecule (target 2) for CRISPR Cascade in the case of RNP2. Additionally, Cas13a can cleave a target nucleic acid molecule that is single stranded while also performing trans-cleavage or collateral cleavage of non-targeted ssRNA upon activation of the RNP complex. In some embodiments, Cas13a RNA guided RNA endonuclease forms a first ribonucleoprotein complex, RNP1, with gRNA specific for a target nucleic acid molecule (target 1). In some embodiments, one or more Cas13a RNA guided RNA endonucleases form a second ribonucleoprotein complex, RNP2, with high K_{D} gRNA specific for signal amplification target nucleic acid molecule (target 2). The specificity of high K_{D} gRNA is discussed below. In some embodiments, Cas13a RNA guided RAN endonucleases form both RNP1 and RNP2 complexes.

### Guide RNAs

As used herein, “single guide RNA,” “guide RNA (gRNA),” “guide sequence” and “sgRNA” can be used interchangeably herein and refer to a single RNA species capable of directing RNA-guided endonuclease mediated cleavage of target nucleic acid molecule (target 1).

The gRNA and sgRNA as used herein refer to a single molecule comprising at least a crRNA region. The crRNA region of the gRNA is a customizable component that enables specificity in every CRISPR reaction. A guide RNA used in the systems and methods can also comprise an endoribonuclease recognition site (e.g., Csy4) for multiplex processing of gRNAs. If an endoribonuclease recognition site is introduced between neighboring gRNA sequences, more than one gRNA can be transcribed in a single expression cassette. Direct repeats can also serve as endoribonuclease recognition sites for multiplex processing.

A gRNA can comprise any polynucleotide sequence having sufficient complementarity with a target sequence (e.g., target 1 and/or target 2) to hybridize with the target sequence and to direct sequence-specific binding of an RNP complex comprising the gRNA and a CRISPR effector protein, such as Cas12a, to the target sequence. A gRNA contains a spacer. The spacer can comprise a plurality of bases that are complementary to the target sequence (such as target 1 or target 2). For example, a spacer can contain about 2, 3, 4, 5, 6, 7, 8, 9, 10, 11, 12, 13, 14, 15, 20, 25, 30, 35, 40, 45, 50, 55, 60, or more bases. The portion of the target sequence that is complementary to the guide sequence is known as the protospacer. When a gRNA molecule is specific for a target sequence (e.g., target 1 or target 2), the gRNA spacer pairs with a portion of the target sequence called the protospacer. The protospacer is the section of the target sequence (e.g., target 1 or target 2) that will be cut. The protospacer located next to a PAM sequence. The PAM sequence can be, for example, 5′-TTTV-3′ for Cas12a, 5′-TTN-3′ for Cas12b, (where V represents A, G, or C).

In some embodiments, the degree of complementarity between a guide sequence and its corresponding target sequence (e.g., target 1 or target 2), when optimally aligned using a suitable alignment algorithm, is about or more than about 50%, 60%, 75%, 80%, 85%, 90%, 95%, 97.5%, 99%, or more. Optimal alignment can be determined with the use of any suitable algorithm for aligning sequences, non-limiting example of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies; available at novocraft.com), ELAND (Illumina, San Diego, Calif.), SOAP (available at soap.genomics.org.cn), and Maq (available at maq.sourceforge.net).

A guide RNA used in the systems and methods described herein are short, single-stranded polynucleotide molecules about 20 nucleotides to about 300 nucleotides in length. The spacer sequence that hybridizes to a complementary protospacer region of the target sequence (e.g., target 1 or target 2) can be about 14, 15, 16, 17, 18, 19, 20, 25, 30, 35 or more nucleotides in length.

In some embodiments, a gRNAs can be synthetically generated or by making the sgRNA in vivo or in vitro, starting from a DNA template.

In some embodiments, a gRNA that is capable of binding a target sequence (e.g., target 1 or target 2) and binding an RNA-guided DNA or RNA endonuclease protein can be expressed from a vector comprising a type II promoter or a type III promoter.

While in type II CRISPR/Cas systems, such as Cas9, a gRNA has at least two regions, a type V CRISPR system such as Cas12a does not. Type II systems such as Cas9, for example, have a CRISPR RNA (crRNA) or spacer sequence, which is a nucleotide sequence complementary to the target sequence, and a tracr RNA, which serves as a binding scaffold for the RNA-guided endonuclease. In Type II systems, the maturation of crRNA is done by host housekeeping protein RNase III together with the trans-activating crRNA (tracrRNA), which is base paired with the pre-crRNA, in presence of Cas9. In contrast, Cas12a processes its own pre-crRNA into mature crRNAs, without the requirement of a tracrRNA, making it a unique effector protein with both endoribonuclease and endonuclease activities. After the pre-crRNA has been transcribed during the expression stage, Cas12a cuts it 4 nt upstream of the hairpin structures formed by the CRISPR direct repeats, producing intermediate crRNA molecules which undergo further processing in vivo into mature crRNAs.

The method of detecting a target nucleic acid molecule (target 1) in a sample further comprises contacting the sample with gRNA specific for the target nucleic acid molecule (target 1). In some embodiments, Cas RNA guided endonuclease such as Cas12a uses the gRNA to direct the Cas protein such as Cas12a to a protospacer sequence on the target nucleic acid molecule (target 1). In some embodiments, a gRNA molecule can be gRNA specific for the target nucleic acid molecule (target 1). In some embodiments, when the spacer region of the gRNA molecule specific for the target nucleic acid molecule (target 1) hybridizes with the protospacer region of the target nucleic acid molecule (target 1), the RNP1 complex is activated.

In some embodiments, the gRNA molecule can be a high K_{D} gRNA specific for the signal amplification target nucleic acid molecule (target 2), as discussed below. Briefly, the method of detecting a target nucleic acid molecule (target 1) in a sample further comprises contacting the sample with a signal amplification target nucleic acid molecule (target 2) and a high K_{D} guide RNA (gRNA) specific for the signal amplification target nucleic acid molecule (target 2). In some embodiments, a Cas RNA guided endonuclease such as Cas12a uses the high K_{D} gRNA to direct the Cas RNA guided endonuclease such as Cas12a to a protospacer sequence on the signal amplification target nucleic acid molecule (target 2). In some embodiments, when the spacer region of the high K_{D} gRNA molecule specific for the signal amplification target nucleic acid molecule (target 2) hybridizes with the protospacer region of the signal amplification target nucleic acid molecule (target 2), an RNP2 complex is activated.

High K_{D} gRNA

A high K_{D} gRNA is specific for the signal amplification target nucleic acid molecule (target 2) and/or the target nucleic acid that is to be detected by an assay. High K_{D} gRNAs can be specific for target 1, target 2, or both target 1 and target 2. High K_{D} gRNA has low binding affinity and can optionally prevent RNP, such as RNP1 or RNP2, complex formation. Any of the K_{D} gRNAs described herein can be used in any method described herein.

In some embodiments a high K_{D} gRNA comprises (i) a portion that prevents formation of an active RNP complex, (ii) a direct repeat, handle, or pseudoknot portion that is active for a Cas enzyme, and (iii) a spacer region with homology to a target nucleic acid molecule.

### Direct Repeat, Handle, or Pseudoknot Portion

gRNAs for Cas (e.g., Cas12a, Cas12b, Cas12c, Cas12d, Cas12e, Cas14, Cas12h, Cas12i, Cas12j, Cas13a, or Cas12g) comprise a direct repeat, handle, or pseudoknot region, herein after referred to as a direct repeat portion. A direct repeat portion of a gRNA interacts with the Cas protein and can direct internalization of the gRNA by a Cas protein. These portions can comprise, for example:

SEQ ID NO: 1
5′ AAUUUCUACUGUUGUAGAU 3′ for Cas12
SEQ ID NO: 2
5′ AAUUUCUACUAUUGUAGAU 3′ for Cas12
SEQ ID NO: 3
5′ AUUUUUGUGCCCAUCGUUGGCAC 3′ for Cas12
SEQ ID NO: 4
5′GAUUUAGACUCCCCAAAA 3′ for Cas 13
SEQ ID NO: 5
5′GAUUUAGAACCCAAAA 3′for Cas13
SEQ ID NO: 6
5′GUUGCAGAACCCGAAUAGACGAAUGAAGGAAUGCAAC 3′ for
Cas14

Direct repeat portions for Cas14 (also known as Cas12f) are also described in, for example, Programmed DNA destruction by miniature CRISPR-Cas14 enzymes, Harrington et al., Science, 362:839 (2018); Xiao et al., Structural basis for substrate recognition and cleavage by the dimerization-dependent CRISPR-Cas12f nuclease, Nucleic Acids Research, 49:4120 (2021).

The direct repeat portions are not limited to these examples as others are available to those of skill in the art. A direct repeat portion can be about 15, 16, 17, 18, 19, 20, 21, 22, 23, 24, 25, 26, 27, 28, 29, 30, 31, 32, 33, 34, 35, 36, 37, 38, 39, 40 or more nucleotides.

### Spacer Portion

A high K_{D} gRNA comprises a spacer portion comprises ribonucleotides that are homologous to a target sequence. A spacer sequence is capable of hybridizing with a target sequence in a target nucleic acid molecule. The spacer portion has about 17, 18, 19, 20, 21, 22, 23, 24, 25, 26 or more nucleotides that are homologous to a target nucleic acid molecule. The spacer portion can be about 95, 96, 97, 98, 99, or 100% homologous to a portion of the target nucleic acid molecule.

Portion that Prevents Formation of an Active RNP Complex

A high K_{D} gRNA additionally comprises a portion that prevents formation of an active RNP complex and/or that is cleavable by an RNA guided DNA or RNA nuclease. This is an extra portion of the high K_{D} guide on the 5′ or 3′ end of the gRNA (made up of the direct repeat portion and the spacer portion). The portion that prevents formation of an active RNP complex can be used to make the high K_{D} gRNA into a chimeric molecule, that is a molecule comprising both RNA and DNA nucleotides. In an embodiment, a high K_{D} gRNA can comprise about 2, 3, 4, 5, 6, 7, 8, 9, 10, 15, 20, 25, 30, 35, 40, 45, 50, 55, 60, 65, 70, 75, 80, or more deoxyribonucleotides and about 1, 2, 3, 4, 5, 6, 7, 8, 9, 10, 15, 20, 25, 30, 35, 40, or 45 ribonucleotides (i.e., the direct repeat portion and spacer portion). The deoxyribonucleotides can prevent formation of an active RNP complex. The deoxyribonucleotides can have one or more secondary structures (e.g., bulge, stem, loop, pseudoknot). In some embodiments, the deoxyribonucleotides can be GC rich (e.g., about 60, 70, 80, 90% G+C content over 10, 15, 20, 25, 30, 35, 40, 45, 50, 55, 60, 65, 70, 75, 80, or more deoxyribonucleotides of the portion that prevents formation of an active RNP complex.

In some embodiments, a high K_{D} gRNA comprises no deoxyribonucleotides, but is otherwise protected from nuclease activity by the use of one or modified bases such as phosphorothioate bases or by the use of other blocking mechanisms such as steric blocking of the nuclease. In some embodiments the portion that prevents formation of an active RNP complex is comprised of ribonucleotides (e.g., when using an RNA guided RNA endonuclease).

In some embodiments, the portion that prevents formation of an active RNP complex is cleavable and when cleaved (by for example an activated Cas protein with indiscriminate single stranded cleavage activity) converts the high K_{D} gRNA to a low K_{D} gRNA. That is, the cleavage of the deoxyribonucleotide portion or the ribonucleic portion by can remove the ability of the high K_{D} gRNA to prevent RNP complex formation. The cleavage can also release or produce a detectable signal. The signal can indicate the presence of target nucleic acid molecules.

High K_{D} gRNA Structures

In some embodiments the structure of a high K_{D} gRNA comprises from 5′ to 3′ a direct repeat portion, a spacer portion, and a portion that prevents formation of an active RNP complex. In some embodiments the structure of a high K_{D} gRNA comprises from 5′ to 3′ a portion that prevents formation of an active RNP complex, a direct repeat portion, and a spacer portion.

A high K_{D} gRNA comprising a direct repeat, handle, or pseudoknot sequence of about 18-40 ribonucleotides, a spacer portion having homology to a target nucleic acid sequence of about 17-26 ribonucleotides, and a portion that prevents formation of an active RNP complex of about 2 to about 50 deoxyribonucleotides.

In some embodiments a high K_{D} gRNA molecule can comprise (i) a gRNA direct repeat portion of about 15 to about 40 ribonucleotides, (ii) a spacer portion of about 17 to about 26 ribonucleotides having homology to a target nucleic acid molecule, and (iii) a portion that prevents formation of an active ribonucleoprotein (RNP) complex of about 2 to about 80 single stranded deoxyribonucleotides. The high K_{D} gRNA can comprise one or more detectable labels. The direct repeat portion and the spacer portion can comprise one or more modified internucleoside linkages (e.g., about 1, 2, 3, 4, 5, 10, 20, 30, 40, 50, 60 or more) configured to provide nuclease resistance to those portions. The one or more modified internucleoside linkages can be phosphorothioate internucleoside linkages.

A high K_{D} gRNA molecule can comprise (i) a gRNA direct repeat portion of 15 to 40 ribonucleotides, (ii) a spacer portion of about 17 to 26 ribonucleotides with homology to a target nucleic acid molecule, (iii) a portion that prevents formation of an active ribonucleoprotein (RNP) complex of 2 to 80 (e.g., about 1, 2, 3, 4, 5, 10, 20, 30, 40, 50, 60, 70, 75, or more) single stranded ribonucleotides, wherein the gRNA direct repeat portion and/or the spacer portion comprise one or more modified internucleoside linkages configured to provide nuclease resistance to those portions. The direct repeat portion can comprise 2, 5, 10, 15, 16, 17, 18, 19, 20, 25, 30, 35, 40, or more internucleoside linkages. The spacer portion can comprise 2, 4, 6, 8, 10, 15, 17, 20, 25, 26, or more internucleoside linkages. The combination of the direct repeat portion and spacer portion can comprise about 5, 10, 20, 30, 40, 50, 60, 70, 80, 90% or more internucleoside linkages. The one or more modified internucleoside linkages can be phosphorothioate internucleoside linkages. The high K_{D} gRNA molecule of can further comprise one or more detectable labels.

A high K_{D} gRNA molecule can comprise a circular molecule (e.g., the 5′end and the 3′end are linked, connected, or otherwise associated with each other). A molecule can comprise (i) a gRNA direct repeat portion of 15 to 40 ribonucleotides, (ii) a spacer portion of 17 to 26 ribonucleotides having homology to a target nucleic acid molecule, and (iii) a portion that prevents formation of an active ribonucleoprotein (RNP) complex of 2 to 80 single stranded deoxyribonucleotides. The portion that prevents formation of an active ribonucleoprotein (RNP) complex can be cleavable by a Cas enzyme having RNA guided DNA endonuclease activity and indiscriminate single stranded deoxyribonucleotide cleavage activity. The direct repeat portion and the spacer portion can comprise one or more modified internucleoside linkages (e.g., about 1, 2, 3, 4, 5, 10, 20, 30, 40, 50, 60 or more) configured to provide nuclease resistance to those portions. The one or more modified internucleoside linkages can be phosphorothioate internucleoside linkages. The high K_{D} gRNA circular molecule can further comprise one or more detectable labels.

A high K_{D} gRNA circular molecule can comprise a gRNA direct repeat portion of 15 to 40 ribonucleotides, (ii) a spacer portion of 17 to 26 ribonucleotides having homology to a target nucleic acid molecule, and (iii) a portion that prevents formation of an active ribonucleoprotein (RNP) complex of 2 to 80 single stranded ribonucleotides, wherein the gRNA direct repeat portion and the spacer portion comprise one or more modified internucleoside linkages (e.g., about 1, 2, 3, 4, 5, 10, 20, 30, 40, 50, 60 or more) configured to provide nuclease resistance to those portions. The portion that prevents formation of an active ribonucleoprotein (RNP) complex can be cleavable by a Cas enzyme having RNA guided RNA endonuclease activity and indiscriminate single stranded ribonucleotide cleavage activity. The one or more modified internucleoside linkages can be phosphorothioate internucleoside linkages. The high K_{D} gRNA circular molecule can further comprise one or more detectable labels.

A high K_{D} gRNA molecule can comprise (i) a gRNA direct repeat portion of about 15 to about 40 ribonucleotides, (iii) a spacer portion of about 17 to about 26 ribonucleotides with homology to a target nucleic acid molecule, (iii) a single stranded deoxyribonucleotide molecule hybridized to the gRNA direct repeat portion and the spacer region, wherein the single stranded deoxyribonucleotide molecule has 3, 4, 5, 6, 7, 8 or more single stranded bulges of 2 or more deoxyribonucleotides, wherein the single stranded bulges can be cleaved by indiscriminate Cas enzyme mediated cleavage, and (iv) one or more detectable labels.

The single stranded bulges are deoxyribonucleotides that are not paired to the gRNA direct repeat portion and the spacer region and therefore bulge out from the gRNA direct repeat portion and the spacer region strand. Other than the bulges, the rest of the single stranded deoxyribonucleotide molecule can be hybridized to the gRNA direct repeat portion and the spacer region with about 70, 80, 90. 95, 96, 97, 98, 99, or 100% homology. In an embodiment the single stranded deoxyribonucleotide molecule has 1, 2, 3, 4, 5, 6, 7, 8, 9, 10 or more additional non-hybridized bases that extend past the 5′ and/or 3′ end of the gRNA direct repeat portion and the spacer region. When the single stranded bulges are cleaved by a Cas enzyme, the pieces of the single stranded deoxyribonucleotide molecule de-hybridizes from the gRNA direct repeat portion and the spacer region such that the gRNA becomes a low K_{D} gRNA molecule. The gRNA direct repeat portion and the spacer portion can comprise one or more modified internucleoside linkages configured to provide nuclease resistance to those portions. The single stranded bulges are cleavable by a Cas enzyme having indiscriminate single stranded cleavage activity.

A high K_{D} gRNA molecule can comprise (i) a gRNA direct repeat portion of 15 to 40 ribonucleotides, (iii) a spacer portion of 17 to 26 ribonucleotides with homology to a target nucleic acid molecule, (iii) a single stranded ribonucleotide molecule hybridized to the gRNA direct repeat portion and the spacer region, wherein the single stranded ribonucleotide molecule has 2, 3, 4, 5, 6, 7, 8 or more single stranded bulges of 2 or more ribonucleotides, wherein the single stranded bulges can be cleaved by indiscriminate Cas enzyme mediated cleavage, and (iv) one or more detectable labels. The single stranded bulges are ribonucleotides that are not paired to the gRNA direct repeat portion and the spacer region and therefore bulge out from the gRNA direct repeat portion and the spacer region strand. Other than the bulges, the rest of the single stranded ribonucleotide molecule can be hybridized to the gRNA direct repeat portion and the spacer region with about 70, 80, 90. 95, 96, 97, 98, 99, or 100% homology. In an embodiment the single stranded ribonucleotide molecule has 1, 2, 3, 4, 5, 6, 7, 8, 9, 10 or more additional non-hybridized bases that extend past the 5′ and/or 3′ end of the gRNA direct repeat portion and the spacer region. When the single stranded bulges are cleaved by a Cas enzyme, the pieces of the single stranded ribonucleotide molecule de-hybridizes from the gRNA direct repeat portion and the spacer region such that the gRNA becomes a low K_{D} gRNA molecule. The gRNA direct repeat portion and the spacer portion can comprise one or more modified internucleoside linkages configured to provide nuclease resistance to those portions. The single stranded bulges can be cleavable by a Cas enzyme having indiscriminate single stranded cleavage activity.

In some embodiments, the high K_{D} gRNA molecule having bulges has one or more reporters and quenchers embedded for detectable signal generation as discussed below.

In some embodiments, the high K_{D} gRNA can be a chimeric molecule. In some embodiments, a high K_{D} gRNA can comprise about 2, 3, 4, 5, 6, 7, 8, 9, 10, 15, 20, 25, 30, 35, 40, 45, 50, 55, 60, 65, 70, 75, 80, or more deoxyribonucleotides and about 1, 2, 3, 4, 5, 6, 7, 8, 9, 10, 15, 20, 25, 30, 35, 40, or 45 ribonucleotides. In some embodiments, about 25 of the ribonucleotides are for Cas (e.g., Cas12a, Cas12b, Cas12c, Cas12d, Cas12e, Cas14, Cas12h, Cas12i, Cas12j, Cas13a, or Cas12g) internalization and about 20 of the ribonucleotides are homologous to a target molecule or a signal amplification target nucleic acid molecule (target 2).

In some embodiments, 2 or more (e.g., about 2, 3, 4, 5, 6, 7, 8, 9, 10, 20, 30, 40, 50, 60, 70, 80 or more) of the deoxyribonucleotides have one or more secondary structures. In some embodiments, the deoxyribonucleotides are GC rich. In some embodiments, the high K_{D} gRNA comprises 2 or more deoxyribonucleotides and about 45 ribonucleotides, wherein about 25 of the ribonucleotides are for Cas (e.g., Cas12a, Cas12b, Cas12c, Cas12d, Cas12e, Cas14, Cas12h, Cas12i, Cas12j, Cas13a, or Cas12g) internalization and about 20 of the ribonucleotides are homologous to the signal amplification target DNA molecule (target 2).

In some embodiments, the 5′ end and 3′ end of the high K_{D} gRNA can be covalently linked to form a circular molecule. In some embodiments, circular gRNA has connecting and cleavable DNA molecules. The circular gRNA design sterically blocks the molecule from internalization into Cas enzyme (e.g., Cas12a, Cas12b, Cas12c, Cas12d, Cas12e, Cas14, Cas12h, Cas12i, or Cas12j) and thus prevents formation of RNP complex. The DNA portion of the gRNA will be cleavable only by trans-cleavage of other activated RNP complexes in the reaction. For example, in some embodiments, activated RNP1 complex can cleave the DNA portion of a circular gRNA molecule by trans-cleavage. After the cleaving of the DNA portion, the circular gRNA molecule converts to a linear gRNA molecule. The linear gRNA molecule then exhibits low K_{D} and is not sterically blocked from internalization into Cas enzyme (e.g., Cas12a, Cas12b, Cas12c, Cas12d, Cas12e, Cas14, Cas12h, Cas12i, or Cas12j). In some embodiments, the linear gRNA can bind to Cas12a, Cas12b, Cas12c, Cas12d, Cas12e, Cas14, Cas12h, Cas12i, or Cas12j enzyme and form an RNP2 complex.

According to some embodiments, the endonuclease activity of activated RNP1 converts the high K_{D} gRNA specific for the signal amplification target nucleic acid molecule (target 2) to form low K_{D} gRNA for subsequent RNP2 formation. In some embodiments, the low K_{D} gRNA resulting from trans-cleavage of the circular high K_{D} gRNA molecule is a linear molecule resembling the gRNA of the Cas enzyme (e.g., Cas12b, Cas12c, Cas12d, Cas12e, Cas14, Cas12h, Cas12i, or Cas12j). In some embodiments, the resulting low K_{D} gRNA is a linear molecule resembling the native, unmodified gRNA of the Cas enzyme (e.g., Cas12b, Cas12c, Cas12d, Cas12e, Cas14, Cas12h, Cas12i, or Cas12j) such as those of Cas12a described in Hewes, Molecular Therapy-Nucleic Acids, 20, (2020): 568-579.

Low K_{D} values of low K_{D} gRNA can be about 1 nM to about 10 nM (e.g., about 1, 2, 3, 4, 5, 6, 7, 8, 9, 10 nM or lower or any range between about 1 nM to about 10 nM). High K_{D} values can be about 10, 100, 1000, or more times higher as compared to low K_{D} values.

In some embodiments, the high K_{D} gRNA is not chimeric and can form an RNP complex with a Cas enzyme for RNA-guided RNA endonuclease activity such as Cas13a or Cas12g. In some embodiments, the high K_{D} gRNA is non-chimeric and linear. In some embodiments, the high K_{D} gRNA is non-chimeric and circular. In some embodiments, the molecular switch can be a portion of the circular high K_{D} gRNA that is protected from nuclease activity. In some embodiments, a portion of the high K_{D} gRNA can be protected from nuclease activity by the use of modified bases such as phosphorothioate bases or by the use of other blocking mechanisms such as steric blocking of the nuclease. Phosphorothioate internucleoside linkages have one of the non-bridging phosphate oxygen atoms replaced with a sulfur atom. Other internucleoside linkages include thiophosphate linkages and those listed in Clave et al., Modified Internucleoside Linkages for Nuclease Resistant Oligonucleotides, RSC Chemical Biology 2: 94 (2021). In some embodiments, the portion of the high K_{D} gRNA that is not protected from nuclease activity, the remaining RNA portion of the high K_{D} gRNA can be cleavable. As such, the cleavage can for convert the high K_{D} gRNA molecule to a low K_{D} molecule.

In some embodiments a high K_{D} gRNA can be designed using an online tool (e.g., CRISPR Design or CHOPCHOP, Broad Institute GPP sgRNA Designer, Benchling CRISPR Guide RNA Design tool, E-CRISP, Synthego Design Tool) that detect PAM sequences and list possible gRNA sequences within a specific target region. These algorithms also predict off-target effects elsewhere in the genome, allowing a user to choose the most specific gRNA for each application. These tools are described in Cui et al., Review of CRISPR/Cas9 sgRNA Design Tools, Interdisciplinary Sci: Computational Life Sci. 10:455 (2018). That is, once a target nucleic acid molecule and a Cas nuclease is selected a user can design a suitable gRNA.

### Signal Amplification Target Nucleic Acid Molecule (Target 2)

Methods of detecting a target nucleic acid molecule in a sample can comprise contacting the sample with a signal amplification target nucleic acid molecule (target 2). In some embodiments, the signal amplification target nucleic acid molecule (target 2) can be a double-stranded DNA molecule for signal amplification or a single-stranded DNA molecule for signal amplification. In some embodiments, the signal amplification target nucleic acid molecule (target 2) can also be single stranded, with or without nuclease resistant base modifications. In some embodiments, the signal amplification target nucleic acid molecule (target 2) can be a single-stranded RNA molecule for signal amplification. In some embodiments, the signal amplification target nucleic acid molecule (target 2) can have nuclease resistant base modifications. For example, one or more modified internucleoside linkages configured to provide nuclease resistance. The one or more modified internucleoside linkages can be, e.g., phosphorothioate internucleoside linkages. In some embodiments, the signal amplification target nucleic acid molecule (target 2) can be a target for activation of RNP2 complex. In some embodiments, the length of the signal amplification target nucleic acid molecule (target 2) can be greater than about 16 bases to activate trans-cleavage of Cas12a enzymes. In some embodiments, the signal amplification target nucleic acid molecule (target 2) can be about 16, 17, 18, 19, 20, 21, 22, 23, 24, 25, 30, 35, 40, 45, 50, 55, 60, 65, 70, 75, 80, 85, 90, 95, or more bases. In some embodiments, the signal amplification target nucleic acid molecule (target 2) has reporter and quencher embedded for signal generation as discussed below. The reporter and quencher can be about 20-30 bases apart (e.g., about 20, 21, 22, 23, 24, 25, 26, 27, 28, 29, 30, or more bases apart) for effective quenching via FRET. In some embodiments, the signal amplification target nucleic acid molecule (target 2) can be synthetic. In some embodiments the signal amplification target nucleic acid molecule (target 2) can be complementary to the about 20 of the ribonucleotides of the high K_{D} gRNA. In some embodiments, the signal amplification target nucleic acid molecule (target 2) is specifically not complementary to the target nucleic acid molecule (target 1) or the gRNA specific for the target nucleic acid molecule (target 1).

### Methods of Detection

In an embodiment, a target nucleic acid molecule can be detected in a sample. A method can comprise contacting the sample with (i) a preassembled complex comprising at least one guide RNA (gRNA) that is specific for the target nucleic acid molecule and a Cas protein having RNA guided DNA or RNA endonuclease activity and indiscriminate ssDNA or ssRNA endonuclease activity and (ii) a high K_{D} guide RNA (gRNA) that can be cleaved by the Cas protein after the preassembled complex is activated by the target nucleic acid molecule, wherein after cleavage, the high K_{D} guide RNA (gRNA) is (i) converted to a low K_{D} guide RNA (gRNA). In an embodiment the high K_{D} guide RNA (gRNA) molecules are detectably labeled and can generate a detectable signal upon cleavage, whereby the target nucleic acid molecule is detected. Any of the high K_{D} guide RNA (gRNA) molecules described herein can be used in this method.

In another embodiment, a CRISPR Cascade mechanism begins with an initial reaction composition containing a pre-assembled RNP1 complex, high K_{D} gRNA, and signal amplification target nucleic acid molecule (target 2) for potential formation of RNP2 complex. In some embodiments, the RNP1 complex is pre-assembled. In some embodiments, the Cas enzyme and gRNA specific for the target nucleic acid molecule (target 1) are not assembled in the initial reaction composition. As discussed above, the RNP1 complex comprises the Cas enzyme and a guide RNA specific to the target nucleic acid molecule (target 1). Furthermore, an RNP1 complex is activated by the target nucleic acid molecule of interest (target 1).

In some embodiments, the RNP1 complex can bind to the target nucleic acid molecule (target 1) and the RNP1 complex can be activated. In some embodiments, the activated RNP1 complex can then begin indiscriminate, or collateral cleavage or trans-cleavage, of single stranded DNA (ssDNA). In some embodiments, the activated RNP1 complex can then begin indiscriminate, or collateral cleavage or trans-cleavage, of single stranded RNA (ssRNA). In some embodiments, the RNP1 complex does not bind to the target nucleic acid molecule (target 1) and the high K_{D} gRNA prevents RNP2 complex formation.

In some embodiments, when the RNP1 complex is activated, Cas RNA guided endonucleases cleave high K_{D} gRNA molecules to release a low K_{D} gRNA molecule. Any of the high K_{D} guide RNA (gRNA) molecules described herein can be used in this method. In some embodiments, the high K_{D} gRNA can be cleaved by the activated RNP1 complex to form low K_{D} gRNA that can form an RNP2 complex. The low K_{D} gRNA has higher binding affinity compared to the high K_{D} gRNA. In some embodiments, the low K_{D} gRNA can internalize and form an RNP2 complex with a Cas enzyme. In some embodiments, binding of the signal amplification target nucleic acid molecule (target 2) activates the formed RNP2 complex. The activated RNP2 complex can begin a second system collateral trans-cleavage (indiscriminate ssDNA endonuclease activity), cleaving more high K_{D} gRNAs to form more low K_{D} gRNA. In some embodiments, binding of the signal amplification target nucleic acid molecule (target 2) activates the formed RNP2 complex. The activated RNP2 complex can begin a second system for collateral trans-cleavage (indiscriminate ssRNA endonuclease activity) cleaving more high K_{D} gRNAs to form more low K_{D} gRNA. Therefore, the binding of the target nucleic acid molecule (target 1) initiates a cascade mechanism by converting high K_{D} guide RNA molecules to low K_{D} guide RNA molecules. As more low K_{D} gRNAs are formed and internalized, more RNP2 complexes are formed. Activation of more RNP2 complexes can trigger a cascade of RNP2 complex formation and activation.

### Amplification

Advantageously, the cascade methods of detection described herein do not require that the target nucleic acid molecules (target 1) be amplified prior to detection. In some embodiments, however, target nucleic acid molecules (target 1) (RNAs and/or DNAs) can be amplified prior to activating the RNP1 complex. Any suitable RNA or DNA amplification technique may be used. In some embodiments, the RNA or DNA amplification is an isothermal amplification. In some example embodiments, the isothermal amplification may be nucleic-acid sequenced-based amplification (NASBA), recombinase polymerase amplification (RPA), loop-mediated isothermal amplification (LAMP), strand displacement amplification (SDA), helicase-dependent amplification (HDA), or nicking enzyme amplification reaction (NEAR). In some embodiments, non-isothermal amplification methods may be used which include, but are not limited to, PCR, multiple displacement amplification (MDA), rolling circle amplification (RCA), ligase chain reaction (LCR), or ramification amplification method (RAM).

In some embodiments, amplification can be performed before the CRISPR Cascade mechanism method of detecting a target nucleic acid molecule (target 1). In some embodiments, amplification can be performed along with the CRISPR Cascade mechanism method of detecting a target nucleic acid molecule (target 1).

In some embodiments, the sample can be a viral or bacterial sample or a biological sample that has been minimally processed, e.g., only treated with a brief lysis step prior to detection. In some embodiments, minimal processing can include thermal lysis at an elevated temperature to release nucleic acids. Suitable methods are contemplated in U.S. Pat. No. 9,493,736, among other references. Common methods for cell lysis involve thermal, chemical, enzymatic, or mechanical treatment of the sample or a combination of those. In some embodiments, minimal processing can include treating the sample with chaotropic salts such as Guanidine Isothiocyanate or Guanidine HCL. Suitable methods are contemplated in U.S. Pat. Nos. 8,809,519, 7,893,251, among other references. In some embodiments, minimal processing can include contacting the sample with reducing agents such as DTT or TCEP and EDTA to inactivate inhibitors and/or other nucleases present in the crude samples. In other embodiments, minimal processing for biofluids can comprise centrifuging the samples to obtain cell-debris free supernatant before applying the reagents. Suitable methods are contemplated in U.S. Pat. No. 8,809,519, among other references. In still other embodiments, minimal processing can comprise performing DNA/RNA extraction to get purified nucleic acids before applying CRISPR Cascade reagents.

In some embodiments, the target nucleic acid molecule (target 1) can be converted from RNA to cDNA by reverse transcription prior to detection. The synthesis of cDNA from an RNA template, via reverse transcription, produces complementary DNA (cDNA). Reverse transcriptases (RTs) use an RNA template and a short primer complementary to the 3′ end of the RNA to direct the synthesis of the first strand cDNA, which can be used directly as a template for the Polymerase Chain Reaction (PCR). This combination of reverse transcription and PCR (RT-PCR) allows the detection of low abundance RNAs in a sample, and production of the corresponding cDNA, thereby facilitating the cloning of low copy genes. Alternatively, the first-strand cDNA can be made double-stranded using DNA Polymerase I and DNA Ligase. These reaction products can be used for direct cloning without amplification. In this case, RNase H activity, from either the RT or supplied exogenously, is required. For example, for RNA targets, the RNA can be first converted to cDNA by performing a reverse transcription step and then cascade can be performed. This can be performed as a single step or two steps where cDNA is formed first and then the cDNA sample is subject to the CRISPR Cascade method discussed herein.

### Detection

In some embodiments, a signal generation pathway can be used to monitor the CRISPR detection reactions described herein. The signal generation pathway can utilize a reporting system including, for example, (1) a high K_{D} gRNA that comprises a detectable signal moiety; (2) a double-stranded DNA molecule that comprises one or two detectable signal moieties; or (3) one or more single stranded nucleic acid reporter molecules.

A label or detectable label is a moiety that can be attached to a nucleic acid molecule or protein to render the nucleic acid molecule or protein detectable.

A detectable label can generate a signal such that the intensity of the signal is proportional to the amount of bound target. Labeled nucleic acid molecules can be prepared by incorporating or conjugating a label that is directly or indirectly detectable by spectroscopic, photochemical, biochemical, immunochemical, electrical, optical, chemical or other means. Suitable detectable labels include, for example, radioisotopes, fluorophores (e.g., fluorescein isothiocyanate (FITC)), phycoerythrin (PE), cyanine (Cy3), VIC fluorescent dye, FAM (6-carboxyfluorescein) or Indocyanine (Cy5), chromophores, chemiluminescent agents, microparticles, enzymes, magnetic particles, electron dense particles, mass labels, spin labels, haptens, and other suitable labels. Nucleic acid molecules and proteins can be labeled by coupling or physically linking a detectable moiety or by indirect labeling by reactivity with another reagent that is directly labeled.

Many real-time detection chemistries can be used to indicate the presence of labeled nucleic acid molecules. Some detection chemistries depend upon fluorescence indicators that change properties as a result of the PCR process. Among these detection chemistries are DNA binding dyes (such as SYBR® Green) that increase fluorescence efficiency upon binding to double stranded DNA. Other real-time detection chemistries can be used including Foerster resonance energy transfer (FRET), where the fluorescence efficiency of a dye is strongly dependent on its proximity to another light absorbing moiety or quencher. These dyes and quenchers can be attached to a gRNA or high K_{D} gRNA. Among the FRET-based detection chemistries are hydrolysis probes and conformation probes. Hydrolysis probes (such as the TaqMan® probe) use a polymerase enzyme to cleave a reporter dye molecule from a quencher dye molecule attached to a polynucleotide probe. Conformation probes (such as molecular beacons) utilize a dye attached to a polynucleotide, whose fluorescence emission changes upon the conformational change of the polynucleotide hybridizing to the target DNA.

Methods for incorporating detectable labels or moieties into nucleic acid molecules and or probes are known. A nucleic acid molecule, protein, or enzyme as described herein can comprise 1, 2, 3, 4, 5, 6, 7, 8 or more detectable labels. A detectable signal moiety or detectable signal moieties are labels that can be used to detect a molecule. In some embodiments, the molecule can be a high K_{D} gRNA with a detectable signal moiety such that the Cas endonuclease conversion of high K_{D} gRNA to low K_{D} gRNA generates a signal. In some embodiments, the molecule can be a dsDNA molecule, such as signal amplification target nucleic acid molecule (target 2), with one or two detectable signal moieties such that cis-cleavage by activated RNP2 complexes generates a signal. In some embodiments, the molecule can be a ssDNA reporter molecule with a detectable signal moiety such that the indiscriminate trans-cleavage by activated RNP1 and or RNP2 complexes generates a signal.

A detectable moiety, label or reporter can be used to detect a target nucleic acid molecule (target 1) as described herein. Guide RNA molecules (such as the high K_{D} gRNA), dsDNA molecules (such as some embodiments of a signal amplification target nucleic acid molecule (target 2)), and ssDNA reporter molecules can be labeled in a variety of ways, including the direct or indirect attachment of a detectable moiety such as a fluorescent moiety, hapten, colorimetric moiety and the like. Examples of detectable moieties include various radioactive moieties, enzymes, prosthetic groups, fluorescent markers, luminescent markers, bioluminescent markers, metal particles, protein-protein binding pairs, protein-antibody binding pairs and the like. Examples of fluorescent moieties include, but are not limited to, yellow fluorescent protein (YFP), green fluorescence protein (GFP), cyan fluorescence protein (CFP), umbelliferone, fluorescein, fluorescein isothiocyanate, rhodamine, dichlorotriazinylamine fluorescein, cyanines, dansyl chloride, phycocyanin, phycoerythrin and the like. Examples of bioluminescent markers include, but are not limited to, luciferase (e.g., bacterial, firefly, click beetle and the like), luciferin, aequorin and the like. Examples of enzyme systems having visually detectable signals include, but are not limited to, galactosidases, glucorinidases, phosphatases, peroxidases, cholinesterases and the like. Identifiable markers also include radioactive compounds such as 1251, 35S, 14C, or 3H. Identifiable markers are commercially available from a variety of sources. As used herein, the term “fluorescent label” includes a signaling moiety that conveys information through the fluorescent absorption and/or emission properties of one or more molecules. Such fluorescent properties include fluorescence intensity, fluorescence lifetime, emission spectrum characteristics, energy transfer, and the like.

Detection method(s) used will depend on the detectable moiety or moieties used. For example, a radioactive label can be detected using a scintillation counter, photographic film as in autoradiography, or storage phosphor imaging. Where the label is a fluorescent label, it can be detected by exciting the fluorochrome with the appropriate wavelength of light and detecting the resulting fluorescence. The fluorescence can be detected visually, by means of photographic film, by the use of electronic detectors such as charge coupled devices (CCDs) or photomultipliers and the like. Similarly, enzymatic labels can be detected by providing the appropriate substrates for the enzyme and detecting the resulting reaction product. Simple colorimetric labels can be detected by observing the color associated with the label. When pairs of fluorophores are used in an assay, they may have distinct emission patterns (wavelengths) so that they can be easily distinguished. In some embodiments, the signal can be detected by lateral flow assays (LFAs). Lateral flow tests are simple devices intended to detect the presence or absence of a target analyte, such as the gRNA, in a sample. Most commonly these tests are used for medical diagnostics, for home testing, or laboratory use. They are often produced in a dipstick format, and the test sample flows along a solid substrate via capillary action. In some LFA methods, after the sample is applied to the test, it encounters a colored reagent which mixes with the sample and transits the substrate encountering lines or zones which have been pretreated with an antibody or antigen. Depending upon the analytes present in the sample the colored reagent can become bound at the test line or zone. Lateral flow dipsticks have been well used in the art for detection of nucleic acids from a liquid sample. Advantageously, these devices require very little expertise to use and can give results within minutes. LFAs can use nucleic acid molecules conjugated nanoparticles (often gold, e.g., RNA-AuNPs or DNA-AuNPs) as a detection probe, which hybridizes to a complementary target sequence. Gold nanoparticles (AuNP) are typically spherical, ranging in size from about 1 nm to about 400 nm in diameter. The hybridization produces a colorimetric signal indicating the detection of the target nucleic acid sequence. The intense red color of RNA-AuNPs or DNA-AuNPs, for example, provides a platform for colorimetric detection using AuNPs as a signal label to trace hybridization of the target nucleic acid molecule. In some embodiments, the high K_{D} gRNA molecules can be modified with AuNP compatible with lateral flow assays (LFAs) (using biotin, streptavidin, etc.) and the AuNP can be released upon cleavage to be detected via an LFA.

In some embodiments, single stranded nucleic acid reporter molecules such as ssDNA reporter molecules or ssRNA molecules can be introduced to show a signal change proportional to the cleavage rate, which increases with every new activated RNP2 complex over time. In some embodiments, the single stranded nucleic acid reporter molecules can also be embedded into the high K_{D} gRNA for real time reporting of results. In some embodiments, the measured detectable signal can be produced by a fluorescent dye pair. For example, the method of detecting a target nucleic acid molecule (target 1) in a sample using a CRISPR Cascade mechanism described herein can involve contacting the sample with a labeled detection ssDNA containing a fluorescent resonance energy transfer (FRET) pair, a quencher/phosphor pair, or both. In some embodiments, the method can comprise contacting the sample with a labeled detection ssDNA containing a FRET pair. In some embodiments, the method can comprise contacting the sample with a labeled detection ssDNA containing a fluorophore/quencher pair.

FRET is phenomenon wherein excitation of one emissive dye is transferred to another without emission of a photon. A FRET pair consists of a donor chromophore and an acceptor chromophore (where the acceptor chromophore may be a quencher molecule). The emission spectrum of the donor and the absorption spectrum of the acceptor must overlap, and the two molecules must be in close proximity. The distance between donor and acceptor at which 50% of donors are deactivated (transfer energy to the acceptor) is defined by the Förster radius, which is typically 10-100 angstroms. Changes in the emission spectrum comprising FRET pairs can be detected, indicating changes in the number of that are in close proximity (i.e., within 100 angstroms of each other). This will typically result from the binding or dissociation of two molecules, one of which is labeled with a FRET donor and the other of which is labeled with a FRET acceptor, wherein such binding brings the FRET pair in close proximity.

Binding of such molecules will result in an increased emission of the acceptor and/or quenching of the fluorescence emission of the donor. FRET pairs (donor/acceptor) suitable for use include, but are not limited to, EDANS/fluorescein, IAEDANS/fluorescein, fluorescein/tetramethylrhodamine, fluorescein/Cy 5, IEDANS/DABCYL, fluorescein/QSY-7, fluorescein/LC Red 640, fluorescein/Cy 5.5 and fluorescein/LC Red 705. In addition, a fluorophore/quantum dot donor/acceptor pair can be used. EDANS is (5-((2-Aminoethyl)amino)naphthalene-1-sulfonic acid); IAEDANS is 5-({2-[(iodoacetyl)amino]ethyl}amino)naphthalene-1-sulfonic acid); DABCYL is 4-(4-dimethylaminophenyl) diazenylbenzoic acid.

Cy3, Cy5, Cy 5.5, and the like, are cyanines. For example, Cy3 and Cy5 are reactive water-soluble fluorescent dyes of the cyanine dye family. Cy3 dyes are red (^{˜}550 nm excitation, ^{˜}570 nm emission and therefore appear green), while Cy5 is fluorescent in the red region (^{˜}650/670 nm) but absorbs in the orange region (^{˜}649 nm). Alexa Fluor dyes, Dylight, IRIS Dyes, Seta dyes, SeTau dyes, SRfluor dyes and Square dyes can also be used.

In another aspect of FRET, an emissive donor molecule and a nonemissive acceptor molecule (“quencher”) may be employed. In this application, emission of the donor will increase when quencher is displaced from close proximity to the donor and emission will decrease when the quencher is brought into close proximity to the donor. Useful quenchers include, but are not limited to, DABCYL, QSY 7 and QSY 33. Useful fluorescent donor/quencher pairs include, but are not limited to EDANS/DABCYL, Texas Red/DABCYL, BODIPY/DABCYL, Lucifer yellow/DABCYL, coumarin/DABCYL and fluorescein/QSY 7 dye. In some embodiments, the ssDNA reporter can comprise an emissive donor molecule and a quencher such that cleavage of the ssDNA reporter molecule by activated RNP complexes generates signal by displacing the quencher from close proximity to the donor.

### Sample Types

A sample can be any biological sample obtained from an organism or a part thereof, such as a plant, animal, bacteria, and the like. In some embodiments, the biological sample is obtained from an animal subject, such as a human subject. A biological sample is any solid or fluid sample obtained from, excreted by or secreted by any living organism, including, without limitation, single celled organisms, such as bacteria, yeast, protozoans, and amoebas among others, multicellular organisms (such as plants or animals, including samples from a healthy or apparently healthy human subject or a human patient affected by a condition or disease to be diagnosed or investigated, such as an infection with a pathogenic microorganism, such as a pathogenic bacteria or virus). For example, a biological sample can be a biological fluid obtained from, for example, blood, plasma, serum, urine, stool, sputum, mucous, lymph fluid, synovial fluid, bile, ascites, pleural effusion, seroma, saliva, cerebrospinal fluid, aqueous or vitreous humor, or any bodily secretion, a transudate, an exudate (for example, fluid obtained from an abscess or any other site of infection or inflammation), or fluid obtained from a joint (for example, a normal joint or a joint affected by disease, such as rheumatoid arthritis, osteoarthritis, gout or septic arthritis), or a swab of skin or mucosal membrane surface.

### Compositions

Provided herein is a composition comprising Cas RNA guided endonuclease; gRNA specific for a target nucleic acid molecule (target 1), a signal amplification target nucleic acid molecule (target 2); and a high K_{D} gRNA specific for the signal amplification target nucleic acid molecule (target 2).

In some embodiments, the composition provided herein comprises a Cas RNA guided endonuclease such as Cas12a, Cas12b, Cas12c, Cas12d, Cas12e, Cas14, Cas12h, Cas12i, Cas12j, Cas13a, or Cas12g.

In some embodiments, the composition provided herein comprises gRNA specific for a target nucleic acid molecule of interest (target 1). In some embodiments, the gRNA molecule is about 2, 3, 4, 5, 6, 7, 8, 9, 10, 15, 20, 25, 30, 35, 40, 45, 50, 55, or more nucleotides in length. In some embodiments, the gRNA has about 50, 55, 60, 65, 70, 75, 80, 85, 90, 95, 99% complementarity with a sequence on the target nucleic acid molecule (target 1).

In some embodiments, the composition provided herein comprises a signal amplification target nucleic acid molecule (target 2). In some embodiments, the signal amplification target nucleic acid molecule (target 2) can be about 2, 3, 4, 5, 6, 7, 8, 9, 10, 15, 20, 25, 30, 35, 40, 45, 50, 55, or more nucleotides in length. In some embodiments, the signal amplification target nucleic acid molecule (target 2) has about 50, 55, 60, 65, 70, 75, 80, 85, 90, 95, 99% complementarity with its gRNA molecule. In some embodiments, its gRNA molecule is the low K_{D} gRNA molecule resulting from cleavage of the high K_{D} gRNA molecule described herein.

In some embodiments, the composition provided herein comprises high K_{D} gRNA specific for the signal amplification target nucleic acid molecule (target 2), for the target nucleic acid molecule (target 1), or both. In some embodiments, the high K_{D} gRNA can be a chimeric molecule. In some embodiments, the high K_{D} gRNA can comprise about 2, 3, 4, 5, 6, 7, 8, 9, 10, 15, 20, 25, 30, 35, 40, 45, 50, 55, 60, 65, 70, 75, 80, or more deoxyribonucleotides and about 1, 2, 3, 4, 5, 6, 7, 8, 9, 10, 15, 20, 25, 30, 35, 40, or 45 ribonucleotides. In some embodiments, about 25 of the ribonucleotides are for Cas internalization and about 20 of the ribonucleotides are homologous to a signal amplification target nucleic acid molecule (target 2). In some embodiments, 2 or more of the deoxyribonucleotides have one or more secondary structures. In some embodiments, the deoxyribonucleotides are GC rich. In some embodiments, the high K_{D} gRNA comprises 2 or more deoxyribonucleotides and about 45 ribonucleotides, wherein about 25 of the ribonucleotides are for Cas internalization and about 20 of the ribonucleotides are homologous to the signal amplification target nucleic acid molecule (target 2). In some embodiments, the 5′ end and 3′ end of the high K_{D} gRNA can be covalently linked to form a circular molecule.

Circular High K_{D} gRNA

Provided herein is a composition comprising a circular high K_{D} gRNA molecule. Generally, a circular high K_{D} gRNA molecule is an engineered guide molecule with a molecular switch such that in one form the structure of the circular high K_{D} gRNA molecule blocks internalization by a Cas enzyme and in the second form the engineered gRNA molecule allows internalization by a suitable Cas enzyme.

In some embodiments, Cas proteins can be CRISPR/Cas Type V effector proteins as discussed above herein such as Cas12a. In some embodiments, the circular high K_{D} gRNA molecule can be adapted to other Cas proteins with trans-cleavage activities such as Cas12b, Cas12c, Cas12d, Cas12e, Cas14, Cas12h, Cas12i, Cas12j, Cas13a, or Cas12g. In some embodiments, any RNA guided endonuclease with collateral trans-cleavage activity can be used in the CRISPR Cascade mechanism described herein. In some embodiments, other variants of Cas derivatives with different functionalities can be used. In some embodiments, diverse structural and functional circular high K_{D} gRNA molecule designs can be developed that follow that are engineered with a molecular switch wherein in one form the structure of the circular high K_{D} gRNA molecule blocks the internalization and in the second form the engineered gRNA molecule allows internalization.

In some embodiments, the high K_{D} gRNA comprises about 2 or more deoxyribonucleotides and about 45 ribonucleotides, wherein about 25 of the ribonucleotides are for internalization by a Cas protein (i.e., they allow for the gRNA to be internalized by the Cas protein) and about 20 of the ribonucleotides are homologous to a target sequence; wherein the 5′ end and 3′ end of the high K_{D} gRNA are covalently linked to form a circular molecule.

In some embodiments, the DNA portion (DNA bases) of the high K_{D} gRNA is trans-cleavable but the high K_{D} gRNA as a whole is not cleavable under certain buffer conditions. The trans-cleavage rate of RNA bases by activated Cas enzyme, e.g., Cas12a enzyme, is negligible as compared to that of DNA bases. As such, the high K_{D} gRNA as a whole remains protected and only the DNA portion (DNA bases) get cleaved under most buffer conditions. For example, buffer conditions can be, but are not limited to, Na⁺ concentration of about 50, 75, 100, 125, 150 mM or higher and Mg⁺² concentration of about 2 mM, 5 mM, 7 mM, 10 mM or higher. The RNA cleavage rate can be less than 10% of the cleavage rate for ssDNA cleavage in, for example in NaCl₂ concentrations of about 100, 150, 200, 300 mM or higher.

Thus, in some embodiments, the same effect can also be achieved by protecting only the linkage between RNA bases in the high K_{D} gRNA by converting the linkage between RNA bases to nuclease resistant modifications. Nuclease resistant modification can be, for example, phosphorothioate or similar modifications. Accordingly, in some embodiments, RNA-RNA bonds of the high K_{D} gRNA will not be cleavable, but DNA-DNA bonds of the high K_{D} gRNA will be cleavable.

In some embodiments, the circular high K_{D} gRNA is not chimeric and can form an RNP complex with a Cas enzyme for RNA-guided RNA endonuclease activity such as Cas13a. In some embodiments, the molecular switch can be a portion of the circular high K_{D} gRNA that is protected from nuclease activity. In some embodiments, a portion of the high K_{D} gRNA can be protected from nuclease activity by the use of modified bases such as phosphorothioate bases or by the use of other blocking mechanisms such as steric blocking of the nuclease. In some embodiments, the portion of the circular high K_{D} gRNA that is not protected from nuclease activity, the remaining RNA portion of the circular high K_{D} gRNA can be cleavable. As such, the cleavage can for convert the high K_{D} gRNA molecule to a low K_{D} molecule.

Compositions and methods are more particularly described below and the Examples set forth herein are intended as illustrative only, as numerous modifications and variations therein will be apparent to those skilled in the art. The terms used in the specification generally have their ordinary meanings in the art, within the context of the compositions and methods described herein, and in the specific context where each term is used. Some terms have been more specifically defined herein to provide additional guidance to the practitioner regarding the description of the compositions and methods.

As used herein, the term “and/or” includes any and all combinations of one or more of the associated listed items. As used in the description herein and throughout the claims that follow, the meaning of “a”, “an”, and “the” includes plural reference as well as the singular reference unless the context clearly dictates otherwise. The term “about” in association with a numerical value means that the value varies up or down by 5%. For example, for a value of about 100, means 95 to 105 (or any value between 95 and 105).

All patents, patent applications, and other scientific or technical writings referred to anywhere herein are incorporated by reference herein in their entirety. The embodiments illustratively described herein suitably can be practiced in the absence of any element or elements, limitation or limitations that are specifically or not specifically disclosed herein. Thus, for example, in each instance herein any of the terms “comprising,” “consisting essentially of,” and “consisting of” can be replaced with either of the other two terms, while retaining their ordinary meanings. The terms and expressions which have been employed are used as terms of description and not of limitation, and there is no intention that in the use of such terms and expressions of excluding any equivalents of the features shown and described or portions thereof, but it is recognized that various modifications are possible within the scope of the claims. Thus, it should be understood that although the present methods and compositions have been specifically disclosed by embodiments and optional features, modifications and variations of the concepts herein disclosed can be resorted to by those skilled in the art, and that such modifications and variations are considered to be within the scope of the compositions and methods as defined by the description and the appended claims.

Any single term, single element, single phrase, group of terms, group of phrases, or group of elements described herein can each be specifically excluded from the claims.

Whenever a range is given in the specification, for example, a temperature range, a time range, a composition, or concentration range, all intermediate ranges and subranges, as well as all individual values included in the ranges given are intended to be included in the disclosure. It will be understood that any subranges or individual values in a range or subrange that are included in the description herein can be excluded from the aspects herein. It will be understood that any elements or steps that are included in the description herein can be excluded from the claimed compositions or methods.

In addition, where features or aspects of the compositions and methods are described in terms of Markush groups or other grouping of alternatives, those skilled in the art will recognize that the compositions and methods are also thereby described in terms of any individual member or subgroup of members of the Markush group or other group.

### EXAMPLES

### Example 1

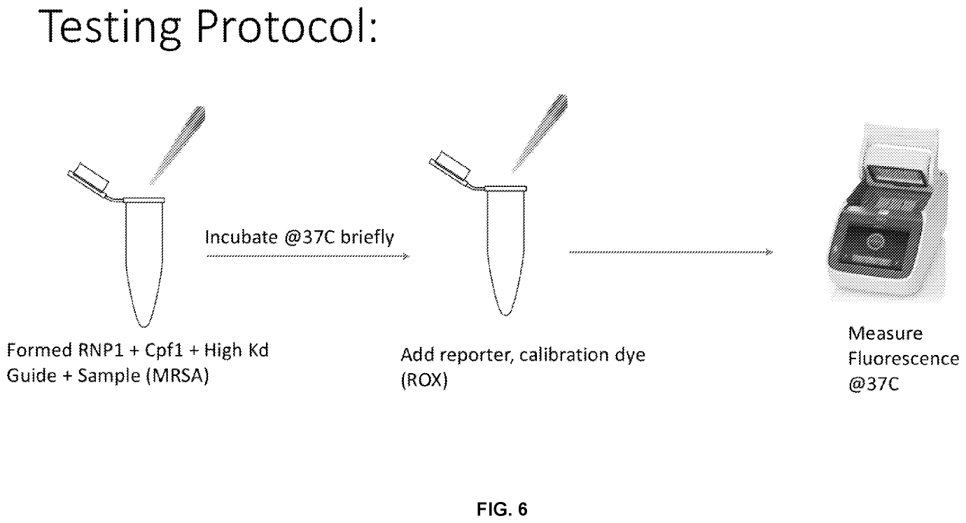

The testing protocol is shown in FIG. 6. The assembled RNP1 complex, Cas12a, and high K_{D} guide were added to a test sample of methicillin-resistant Staphylococcus aureus (MRSA) nucleic acids and incubated briefly at 37° C. A reporter and calibration dye (ROX) were added. Fluorescence was measured at 37° C. A calibration dye is not affected by amplification products. It is affected by anything else that would alter overall fluorescence readings, such as bubbles in wells, evaporation condensation or droplets, instrument issues, such as electrical surges. This allows the calibration dye to serve as a passive reference dye that enables fluorescent normalization for data.

Unless otherwise stated, all reactions were performed in nuclease assay buffer containing: 50 mM NaCl, 10 mM Tris-HCl, 100 μg/ml BSA, PH 7.9 at 25° C. Cas12a enzyme is stored at 100 uM or 10 uM, in Storage Buffer containing: 500 mM NaCl, 20 mM sodium acetate, 0.1 mM EDTA, 0.1 mM TCEP, 50% Glycerol, pH 6 at 25° C.

The reporters were a 5nt base ssDNA sequence with a fluorophore on the 5′ end and a quencher on the 3′ end, and were obtained from (Integrated DNA Technologies), with sequence: 5′: 6-FAM/TTATT/IABKFQ:-3′. All reagents were prepared and stored as aliquots at either −20° C. or −80° C. and thawed right before use in the assay. All oligonucleotides in the assay were stored at 10 uM concentration or higher in T50 buffer (10 mM Tris-Cl, 50 mM NaCl, pH 8.0) and diluted as required before the assay.

### Methods

Step 1: Formation of the RNP1 Complex. For preparation of LbCas12a-crRNA complexes, mature or pre-crRNA constructs were ordered as full RNA sequences with both the spacer sequence and structural sequence specific to LbCas12a from (Integrated DNA Technologies). LbCas12a Enzyme was ordered from NEB Biolabs (M0653T). RNP complexes were formed by incubating CPF1 (cas12a) enzyme with a pre-crRNA containing a spacer compliment to the sample target (MRSA), in nuclease assay buffer containing 15 mM MgCl₂, 275 nM CPF1 enzyme and 250 nM of guide. This formation occurred at room temperature for 20 minutes before addition to subsequent steps.

Step 2: Creation of master mix containing High K_{D} guide, IbCas12a, MRSA, High-KD-guide-complimentary-target. All concentrations refer to the final concentration of components in a 10 uL reaction. This master mix contained 5 nM High K_{D} guide, 50 nM Cas12a, 50 mM NaCl, 5 mM MgCl₂, and varying target concentrations (MRSA). All components except for the sample in this master mix were prepared in bulk in nuclease assay buffer, and 7 uL are added to 1 uL of MRSA target DNA. Typical protocols tested a standard curve of MRSA over 4 orders of magnitude, as well as a negative control containing no target DNA.

Step 3: 2 uL of RNP1 complex (Step 1) was added to 6 ul of master mix (Step 2) and allowed to incubate for 5 minutes at 37° C. in a 384-well microplate. After this incubation, the reaction is paused, and 2 ul of solution containing reporter molecule and calibration dye was added to the 8 uL mixture to yield 500 nM of reporter in the 10 uL reaction. The reaction was placed in a fluorescence plate reading thermocycler and fluorescence levels were measured every minute for 30 minutes at 37° C.

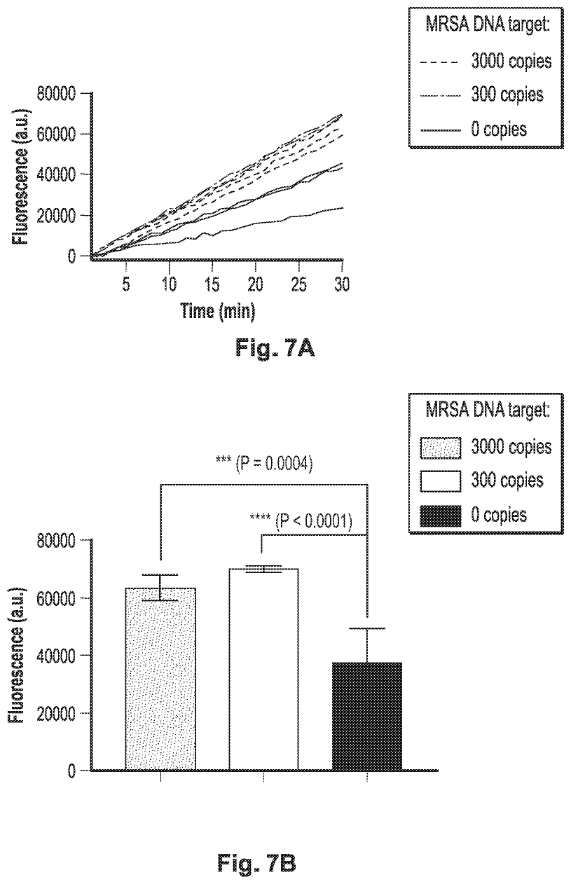

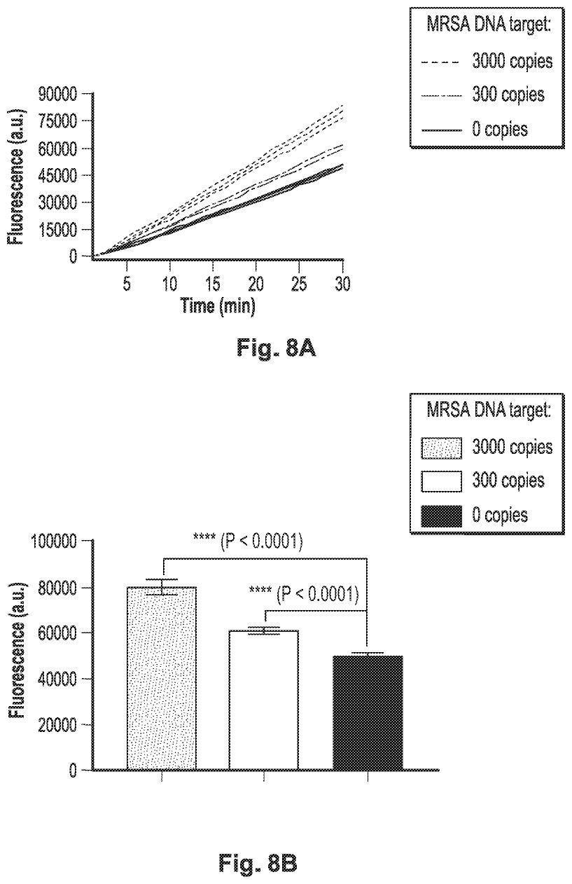

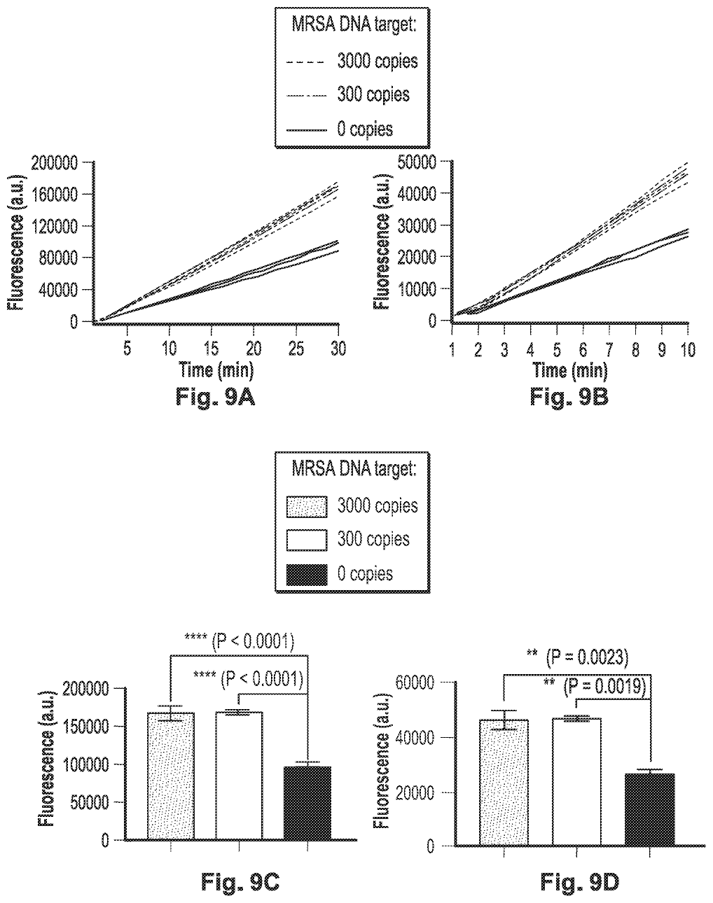

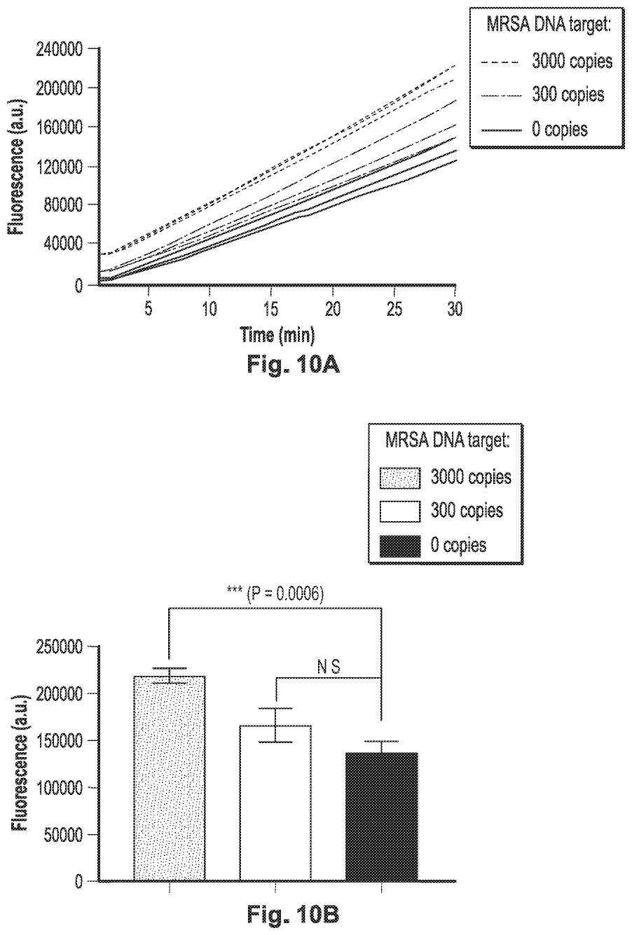

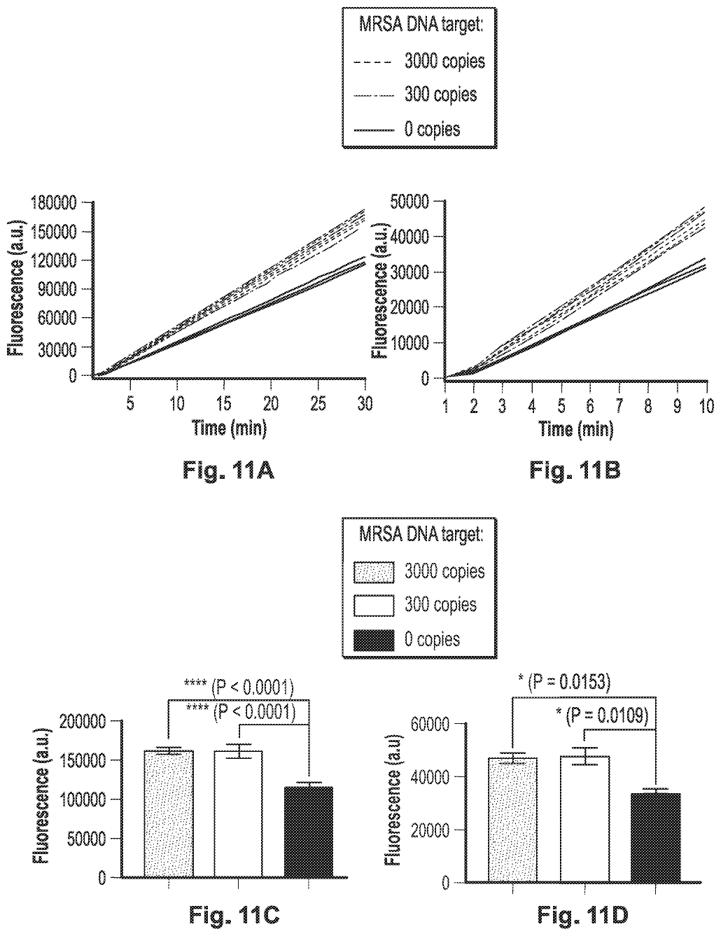

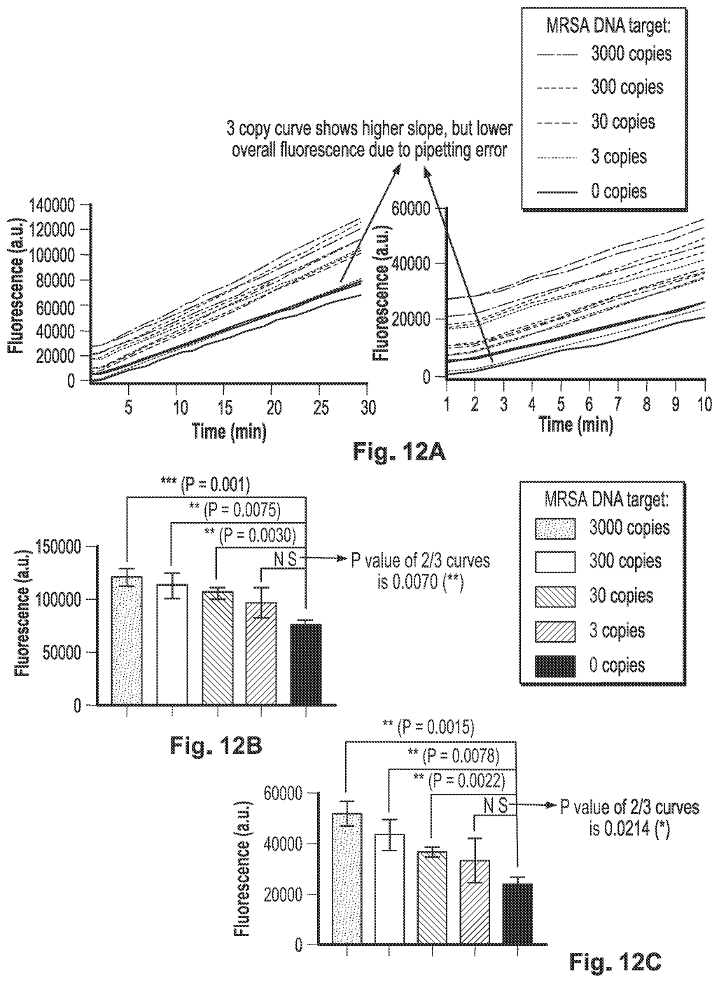

FIGS. 7-12 show the successful detection of 3,000, 300, 30, and 3 copies of the target nucleic acid molecules (MRSA) as compared to a negative control containing no copies of the target nucleic acid molecules.

We claim:

1. A method of detecting a target nucleic acid molecule in a sample comprising contacting the sample with:
(a) Cas RNA guided endonuclease molecules having RNA guided DNA or RNA endonuclease activity and indiscriminate ss RNA or ssDNA endonuclease activity;
(b) guide RNA (gRNA) specific for the target nucleic acid molecule;
(c) a signal amplification target nucleic acid molecule;
(d) a high K_{D} guide RNA (gRNA) specific for the signal amplification target nucleic acid molecule; and
(e) detecting a signal indicating presence of the target nucleic acid molecule.

2. The method of claim 1, wherein the signal amplification target nucleic acid molecule comprises a nuclease resistant signal amplification target nucleic acid molecule.

3. The method of claim 1, wherein the high K_{D} guide RNA (gRNA) specific for the signal amplification target nucleic acid molecule comprises a portion that is protected from nuclease activity.

4. The method of claim 1, wherein the high K_{D} gRNA is configured to be cleaved by a first ribonucleoprotein complex comprising the Cas RNA guided endonuclease and the gRNA specific for the target nucleic acid molecule when the first ribonucleoprotein complex is activated by the target nucleic acid molecule.

5. The method of claim 4, wherein cleavage of the high K_{D} gRNA by the first ribonucleoprotein complex converts the high K_{D} gRNA into a low K_{D} gRNA.

6. The method of claim 5, wherein the low K_{D} gRNA can form a second ribonucleoprotein complex with the Cas RNA guided endonuclease, wherein the second ribonucleoprotein complex can be activated by the signal amplification target nucleic acid molecule.

7. The method of claim 1, wherein the high K_{D} gRNA comprises a detectable signal moiety.

8. The method of claim 1, wherein the signal amplification target nucleic acid molecule comprises one or more detectable signal moieties.

9. The method of claim 1, wherein the sample is further contacted with one or more single stranded nucleic acid reporter molecules.

10. The method of claim 1, wherein the high K_{D} gRNA comprises: about 2 or more deoxyribonucleotides and about 45 ribonucleotides, wherein about 25 of the ribonucleotides are for Cas internalization and about 20 of the ribonucleotides are homologous to the target nucleic acid molecule.

11. The method of claim 10, wherein the about 2 or more deoxyribonucleotides have one or more secondary structures.

12. The method of claim 10, wherein the high K_{D} gRNA comprises about 2 or more deoxyribonucleotides and about 45 ribonucleotides, wherein about 25 of the ribonucleotides are for Cas internalization and about 20 of the ribonucleotides are homologous to the target nucleic acid molecule; wherein a 5′ end and a 3′ end of the high K_{D} gRNA are covalently linked to form a circular molecule; the about 2 or more deoxyribonucleotides are trans-cleavable, and the about 45 ribonucleotides are not trans-cleavable.

13. The method of claim 1, further comprising amplifying the target nucleic acid molecule prior to or along with the steps of claim 1.

14. The method of claim 1, wherein the sample comprises a minimally processed biological sample.

15. The method of claim 1, wherein the target nucleic acid molecule is cDNA.

16. The method of claim 1, wherein the high K_{D} gRNA comprises any high K_{D} gRNA of claims 20-43.

17. The method of claim 1, wherein the Cas RNA guided endonuclease molecules and the guide RNA (gRNA) are pre-assembled.

18. A composition comprising:
(a) Cas RNA guided endonuclease;
(b) gRNA specific for a target nucleic acid molecule;
(c) a signal amplification target nucleic acid molecule; and
(d) a high K_{D} gRNA specific for the signal amplification target nucleic acid molecule.

19. The composition of claim 18, wherein the high K_{D} gRNA comprises any high K_{D} gRNA of claims 20-43.

20. A high K_{D} gRNA molecule comprising (i) a gRNA direct repeat portion of 15 to 40 ribonucleotides, (ii) a spacer portion of 17 to 26 ribonucleotides having homology to a target nucleic acid molecule, (iii) a portion that prevents formation of an active ribonucleoprotein (RNP) complex of 2 to 80 single stranded deoxyribonucleotides.

21. The high K_{D} gRNA molecule of claim 20, further comprising one or more detectable labels.

22. The high K_{D} gRNA molecule of claim 20, wherein the gRNA direct repeat portion and the spacer portion comprise one or more modified internucleoside linkages configured to provide nuclease resistance to those portions.

23. The high K_{D} gRNA molecule of claim 22, wherein the one or more modified internucleoside linkages are phosphorothioate internucleoside linkages.

24. A high K_{D} gRNA molecule comprising (i) a gRNA direct repeat portion of 15 to 40 ribonucleotides, (ii) a spacer portion of 17 to 26 ribonucleotides with homology to a target nucleic acid molecule, (iii) a portion that prevents formation of an active ribonucleoprotein (RNP) complex of 2 to 80 single stranded ribonucleotides, wherein the gRNA direct repeat portion and/or the spacer portion comprise one or more modified internucleoside linkages configured to provide nuclease resistance to those portions.

25. The high K_{D} gRNA molecule of claim 24, wherein the one or more modified internucleoside linkages are phosphorothioate internucleoside linkages.

26. The high K_{D} gRNA molecule of claim 24, further comprising one or more detectable labels.

27. A circular high K_{D} gRNA molecule comprising (i) a gRNA direct repeat portion of 15 to 40 ribonucleotides, (ii) a spacer portion of 17 to 26 ribonucleotides having homology to a target nucleic acid molecule, and (iii) a portion that prevents formation of an active ribonucleoprotein (RNP) complex of 2 to 80 single stranded deoxyribonucleotides.

28. The circular high K_{D} gRNA molecule of claim 27, wherein the portion that prevents formation of an active ribonucleoprotein (RNP) complex is cleavable by a Cas enzyme having indiscriminate single stranded deoxyribonucleotide cleavage activity.

29. The circular high K_{D} gRNA molecule of claim 27, wherein the direct repeat portion and the spacer portion can comprise one or more modified internucleoside linkages configured to provide nuclease resistance to those portions.

30. The circular high K_{D} gRNA molecule of claim 29, wherein the one or more modified internucleoside linkages can be phosphorothioate internucleoside linkages.

31. The high K_{D} gRNA circular molecule of claim 27, further comprising one or more detectable labels.

32. A high K_{D} gRNA circular molecule comprising a gRNA direct repeat portion of 15 to 40 ribonucleotides, (ii) a spacer portion of 17 to 26 ribonucleotides having homology to a target nucleic acid molecule, and (iii) a portion that prevents formation of an active ribonucleoprotein (RNP) complex of 2 to 80 single stranded ribonucleotides, wherein the gRNA direct repeat portion and/or the spacer portion comprises one or more modified internucleoside linkages configured to provide nuclease resistance to those portions.

33. The high K_{D} gRNA circular molecule of claim 32, wherein the portion that prevents formation of an active ribonucleoprotein (RNP) complex is cleavable by a Cas enzyme having indiscriminate single stranded ribonucleotide cleavage activity.

34. The high K_{D} gRNA circular molecule of claim 32, wherein the one or more modified internucleoside linkages are phosphorothioate internucleoside linkages.

35. The high K_{D} gRNA circular molecule of claim 32, further comprising one or more detectable labels.

36. A high K_{D} gRNA molecule comprising (i) a gRNA direct repeat portion of 15 to 40 ribonucleotides, (iii) a spacer portion of 17 to 26 ribonucleotides with homology to a target nucleic acid molecule, (iii) a single stranded deoxyribonucleotide molecule hybridized to the gRNA direct repeat portion and the spacer region, wherein the single stranded deoxyribonucleotide molecule has 3 or more single stranded bulges of 2 or more deoxyribonucleotides, wherein the single stranded bulges can be cleaved by indiscriminate Cas enzyme mediated cleavage, and (iv) one or more detectable labels.

37. The high K_{D} gRNA molecule of claim 36, wherein gRNA direct repeat portion and the spacer portion comprise one or more modified internucleoside linkages configured to provide nuclease resistance to those portions.

38. The high K_{D} gRNA molecule of claim 36, wherein the single stranded deoxyribonucleotide molecule has 3 or more single stranded bulges of 2 or more deoxyribonucleotides.

39. The high K_{D} gRNA molecule of claim 36, wherein the three or more single stranded bulges are cleavable by a Cas enzyme having indiscriminate single stranded cleavage activity.

40. A high K_{D} gRNA molecule comprising (i) a gRNA direct repeat portion of 15 to 40 ribonucleotides, (iii) a spacer portion of 17 to 26 ribonucleotides with homology to a target nucleic acid molecule, (iii) a single stranded ribonucleotide molecule hybridized to the gRNA direct repeat portion and the spacer region, wherein the single stranded ribonucleotide molecule has 3 or more single stranded bulges of 2 or more ribonucleotides, wherein the single stranded bulges can be cleaved by indiscriminate Cas enzyme mediated cleavage, and (iv) one or more detectable labels.

41. The high K_{D} gRNA molecule of claim 40, wherein the gRNA direct repeat portion and the spacer portion comprise one or more modified internucleoside linkages configured to provide nuclease resistance to those portions.

42. The high K_{D} gRNA molecule of claim 40, wherein the single stranded ribonucleotide molecule has 3 or more single stranded bulges of 2 or more ribonucleotides.

43. The high K_{D} gRNA molecule of claim 40, wherein the three or more single stranded bulges are cleavable by a Cas enzyme having indiscriminate single stranded cleavage activity.

44. A method of detecting a target nucleic acid molecule in a sample comprising contacting the sample with (i) a preassembled complex comprising at least one guide RNA (gRNA) that is specific for the target nucleic acid molecule and a Cas protein having RNA guided DNA or RNA endonuclease activity and indiscriminate ssDNA or ssRNA endonuclease activity and (ii) a detectably labeled high K_{D} guide RNA (gRNA) that can be cleaved by the Cas protein after the preassembled complex is activated by the target nucleic acid molecule, wherein after cleavage, the high K_{D} guide RNA (gRNA) is (i) converted to a low K_{D} guide RNA (gRNA); and (ii) generates a detectable signal, whereby the target nucleic acid molecule is detected.

45. The method of claim 44, wherein the high K_{D} guide RNA comprises the high K_{D} guide RNA of any of claims 2-43.

46. The method of claim 44, wherein the low K_{D} guide RNA (gRNA) can bind to a ribonucleoprotein complex.
